(19) United States
(12) Patent Application Publication
(10) Pub. No.: US 2024/0358790 A1
(43) Pub. Date: Oct. 31, 2024

(54) COMPOSITIONS AND METHODS FOR TREATING AUTOIMMUNITY, INCLUDING AUTOIMMUNITY ASSOCIATED WITH CANCER AND CANCER THERAPY

(71) Applicant: Interk Peptide Therapeutics Limited, New South Wales (AU)

(72) Inventor: Michael Agrez, New South Wales (AU)

(73) Assignee: Interk Peptide Therapeutics Limited, New South Wales (AU)

(21) Appl. No.: 18/684,922

(22) PCT Filed: Aug. 19, 2022
(86) PCT No.: PCT/AU2022/050932

§ 371 Date: Feb. 20, 2024

(30) Foreign Application Priority Data
Aug. 20, 2021 (AU) 2021902626

(51) Int. Cl. A61K 38/08; A61K 9/00; A61P 37/04; G01N 33/68

(52) U.S. Cl. CPC A61K 38/08; A61K 9/0019; A61K 9/0053; A61P 37/04; G01N 33/6866; G01N 33/6869; G01N 2333/55; G01N 2333/57; G01N 2333/7155

(57) ABSTRACT

The present invention related to methods and compositions for maintaining IL-2 homeostasis, including low levels CN of IL-2, in a subject, for treating autoimmune disorders. In a preferred embodiment, the composition comprises 2-amino-dodecanoic acid (2Adod)-conjugated peptides selected from RSKAKNPLYR-(2Adod)₂-NH₂, RSKAKNPLYR-(2ADod)₄-NH₂, or peptides with the same sequence where all residues are substituted with D-amino acids, conjugated to two or four 2Adod.

### TECHNICAL FIELD

The present invention related to methods and compositions for maintaining IL-2 homeostasis, including low levels of IL-2, in a subject, for treating autoimmune disorders.

### BACKGROUND OF INVENTION

Autoimmune diseases can be induced by the actions of dysfunctional T cells, B cells and dendritic cells directed towards production of self-antigens that results in tissue destruction. Non-receptor Src family kinase (SFK) members are critical mediators of pro-inflammatory signalling pathways that can contribute to autoimmunity via activation of JAK/STATs and production of IFNg. T cell receptor (TCR) activation and signalling leads to interleukin-2 (IL-2) production and the TCR-associated lymphocytic-specific protein tyrosine kinase, Lck, plays a key role in fine-tuning IL-2 production to avoid either autoimmunity or anergy.

There is a need for modulators of IL-2 homeostasis that provide maintenance of IL-2 at a level to benefit autoimmune diseases.

### SUMMARY OF INVENTION

In one embodiment the present invention provides a method of treating or preventing an autoimmune disorder in a subject, said method comprising administering to the subject a therapeutically effective amount of a peptide comprising an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂.

In another embodiment, the present invention provides a method as described herein, wherein the autoimmune disorder is a disorder associated with dysregulated IL-2 homeostasis.

In a further embodiment the present invention provides a method as described herein, wherein the autoimmune disorder is an IL-2 mediated disorder.

In a further embodiment the present invention provides a method as described herein, wherein the autoimmune disease is associated with dysregulated IL-2 and/or IL-2Ralpha (CD25) production.

In a further embodiment the present invention provides a method as described herein, wherein the subject is deficient in IL-2 and IL-2Ralpha (CD25) production.

In a further embodiment the present invention provides a method as described herein, wherein the autoimmune disorder is selected from the group consisting of allergic asthma, rheumatoid arthritis, multiple sclerosis, systemic lupus erythematosis and other lupus disorders, Type 1 insulin-dependent diabetes mellitus (IDDM), psoriasis, scleroderma, glomerular nephritis, ankylosing spondylitis, and GVHD.

In a further embodiment the present invention provides a method as described herein, wherein the subject has cancer.

In a further embodiment the present invention provides a method as described herein, wherein the subject is receiving cancer therapy.

In a further embodiment the present invention provides a method as described herein, wherein the effective amount of a peptide comprising an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂ modulates the activity of Lck and/or G-protein signalling to maintain homeostatic levels of IL-2 in the subject.

In a further embodiment the present invention provides a method as described herein, wherein the homeostatic levels of IL-2 are produced by cells selected from the group consisting of B cells, T cells and dendritic cells.

In a further embodiment the present invention provides a method as described herein, wherein the effective amount of a peptide comprising an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂ does not induce IFNg and/or IL-12p40.

In a further embodiment the present invention provides a method as described herein, wherein the therapeutically effective amount of a peptide comprising an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂ is administered orally and/or topically.

In a further embodiment the present invention provides a method as described herein, wherein the peptide consists of an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂.

In a further embodiment the present invention provides a use of therapeutically effective amount of a peptide comprising an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂ in a method of treating or preventing an autoimmune disorder in a subject.

In a further embodiment the present invention provides a use of a peptide comprising an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂ in the manufacture of a medicament for treating an autoimmune disorder in a subject.

In a further embodiment the present invention provides an oral dose form comprising an effective amount of a peptide comprising an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂ for the treatment of an autoimmune disorder in a subject.

In a further embodiment the present invention provides a use as described herein, wherein the peptide is administered orally or topically.

In a further embodiment the present invention provides a method as described herein wherein the peptide is administered by injection.

In a further embodiment the present invention provides a method as described herein wherein the peptide is administered in the form of a pharmaceutical composition.

In a further embodiment the present invention provides a method as described herein wherein the pharmaceutical composition is administered to the subject simultaneously or sequentially with a cancer immunotherapy.

### BRIEF DESCRIPTION OF DRAWINGS

FIG. 1: Selective targeting of Src family kinases. Assays were performed as described in Example 1.

FIG. 2: Maintenance of IL-2 homeostasis using RSKAKNPLYR-(2Adod)₄-NH₂. (a)(b): PBMCs were cultured for 24 hrs together with vehicle control (0.13% H₂O), or IK14004 (0.08-1.25 μM), and stimulated with soluble anti-CD3 (1 μg/mL). After 24 hrs, cells were recovered and stained for CD69 and expression assessed by flow cytometry. Data presented as CD69 expression (% and mean fluorescent intensity, MFI) within CD8⁺ cells+SEM, n=4. Cultures treated in the presence of IK14004 (0.31 and 0.63 μM) is represented as an n=3. *p<0.05, **p<0.01, as determined using a mixed-effects analysis with Dunnett's post-test comparing peptide to the vehicle control. The red dotted line represents the mean stimulated media only control value and the blue dotted line represents the mean unstimulated control group value. (c) PBMCs were cultured for 24 hrs together with vehicle control (0.13% H₂O), or IK14004 (0.08-1.25 μM), and stimulated with soluble anti-CD3 (1 μg/mL). After 24 hrs, supernatants were collected and assessed for IL-2 concentration (pg/mL) by ELISA. Data shown as IL-2 concentration (pg/mL)+/−SEM, n=4. No statistical significance as determined using a repeated measures (RM) two-way ANOVA with Dunnett's post-test comparing the test peptide to the vehicle control group. The red dotted line represents the mean stimulated media only control value and the blue dotted line represents the unstimulated control value. IL-2 production fell below the level of detection at the 72 hr timepoint. (d),(e): CD25 expression in CD4 and CD8 positive cells from stimulated PBMCS. Freshly isolated PBMCs were stimulated with anti-CD3 (1 μg/mL) for 24 hrs in the presence of test peptide IK14004 at indicated concentrations (μM). Data are presented as mean+/−SEM from 4 donors. *p<0.05, **p<0.01, ***p<0.001, ****p<0.0001, two-way ANOVA with Dunnett's post-test was used to compare concentrations of each peptide to vehicle (0). (f) CD14+ monocytes were isolated from fresh PBMCs and cultured for 72 hrs in the presence of test peptides over a 5-point concentration curve plus vehicle (0-1.25 μM) and anti-CD3 (1 μg/mL). After 72 hrs, cells were assessed for CD25 expression by flow cytometry. Data presented indicates the mean percentage of positive cells after peptide treatment, +/−SEM, n=4. Data were analysed by RM two-way ANOVA with Dunnett's post-test comparing each peptide concentration with vehicle, ****p<0.0001. (g) Isolated T cells (CD3+) were stimulated with anti-CD3 anti-CD28 Dynabeads™ and cultured together with peptide IK14004 over a 5-concentration range plus vehicle control (0-1.25 μM) for 72 hrs after which supernatants were collected and assessed for IL-2 by ELISA. Data presented shows the mean IL-2 μg/mL+/−SEM, n=4. Data were analysed by RM two-way ANOVA with Dunnett's post-test comparing each peptide concentration with vehicle, **p<0.01, ***p<0.001, ****p<0.0001. (h) The A 12 well-plate was coated with a solution of anti-CD3 (5 μg/ml) made up in PBS (total volume 250 μl/well) and incubated overnight at 37° C. The coating solution was aspirated off and the coated wells gently washed with PBS (twice, 1 ml, 5 minutes). JCaM1.6 cells were seeded at (1×10⁶ cells/well) in the “anti-CD3 coated wells” and subsequently stimulated with anti-CD28 (5 μg/ml), as well as treated with various concentrations of peptide IK14004 (0, 0.625, 1.25 and 2.5 μM) The cells were then incubated for 48 hours at 37° C. The cell suspensions were checked under the microscope, then transferred into 2 ml labelled tubes and centrifuged at 30,000 g for 10 minutes. The supernatant and pellet were separated for each sample and the supernatant (100 μl, n=3) analysed for the IL-2 content using ELISA. (i) CD3+ T cells were cultured for 72 hrs together with vehicle control (0.13% H₂O) or IK14004 (0.08-1.25 μM), or IK14004 (0.08-1.25 μM) in the presence of inhibitor A-770041 (100 nM) and stimulated with soluble anti-CD3 anti-CD28 stimulation beads (4:1 cell to bead ratio). After 72 hrs, cells were recovered and stained for GNA11, and expression assessed by flow cytometry. GNA11 expression was detected using a PE conjugated donkey F(ab′2) anti-rabbit IgG H&L antibody. Data presented as GNA11 expression (mean fluorescence intensity, MFI) within CD4+ T cells, +/−SEM, n=4. *p<0.05, **p<0.01, ***p<0.001 as determined using a repeated measures (RM) two-way ANOVA with Dunnett's post-test comparing peptide to the vehicle control. The red dotted line represents the mean stimulated media only control value and the blue dotted line represents the mean unstimulated control group value. (j) CD3+ T cells were cultured for 72 hrs together with vehicle control (0.1% DMSO), the small molecule inhibitor A-770041 (100 nM), and stimulated with soluble anti-CD3 anti-CD28 stimulation beads (4:1 cell to bead ratio) for 72 hrs. After 72 hrs, supernatants were collected and assessed for IL-2 concentration (pg/mL) by ELISA. Data shown as IL-2 (pg/mL)+/−SEM, n=12. ****p<0.0001, as determined using an unpaired t-test comparing A-770041 to the 0.13% water vehicle control.

FIG. 3: Suppression of pro-inflammatory cytokines by RSKAKNPLYR-(2Adod4). (a) PBMCs were cultured for 72 hrs together with vehicle control (0.13% H₂O), or IK14004 (0.08-1.25 μM), and stimulated with soluble anti-CD3 (1 μg/mL). After 72 hrs, cells were recovered and stained for viability as assessed by flow cytometry. Data presented as % viable cells +/−SEM, n=4. No statistical significance as determined using a repeated measures (RM) two-way ANOVA with Dunnett's post-test comparing peptide to the vehicle control. The red dotted line represents the mean stimulated media only control value and the blue dotted line represents the mean unstimulated control group value. (b) CD3+ T cells were cultured for 72 hrs together with vehicle control or test peptide (0.08-1.25 μM) and stimulated with anti-CD3 anti-CD28 dynabeads at a ratio of 4:1 cells to dynabeads. After 72 hrs, cells were recovered and stained for viability as assessed using flow cytometry. Data presented as % viable cells +/−SEM, n=4; No statistical significance as determined using two-way ANOVA with Dunnett's post-test comparing peptide to the vehicle control. Red dotted line indicates stim only, blue dotted line indicates unstimulated control. (c) Immature monocyte derived DCs (iMoDCs) were derived from isolated CD14+ monocytes cultured for 7 days in Mo-DC differentiation media. iMoDCs were cultured for 72 hrs in the presence of test peptides over a 5-point concentration curve plus vehicle (0-1.25 μM) and anti-CD3 (1 μg/mL). After 72 hrs, cells were assessed for viability by flow cytometry. Data presented indicates the mean percentage of viable cells after peptide treatment, +/−SEM, n=4. Data were analysed by RM two-way ANOVA with Dunnett's post-test comparing each peptide concentration with vehicle, *p<0.05, **p<0.01, ***p<0.001. (d) Immature monocyte derived DCs (iMoDCs) were derived from isolated CD14+ monocytes cultured for 7 days in Mo-DC differentiation media. iMoDCs were cultured for 72 hrs in the presence of test peptides over a 5-point concentration curve plus vehicle (0-1.25 μM) and anti-CD3 (1 μg/mL). After 72 hrs, cells were assessed for expression of intracellular Ki67 by flow cytometry. Data presented indicates the average geometric mean MFI of K167 after peptide treatment, +/−SEM, n=4. Data were analysed by RM two-way ANOVA with Dunnett's post-test comparing each peptide concentration with vehicle, ***p<0.001, ****p<0.0001. (e), (f) Immature monocyte derived DCs (iMoDCs) were derived from isolated CD14+ monocytes cultured for 7 days in Mo-DC differentiation media. iMoDCs were cultured for 72 hrs in the presence of test peptides over a 5-point concentration curve plus vehicle (0-1.25 μM) and anti-CD3 (1 μg/mL). After 72 hrs, supernatants were collected and assessed for IL-12p40 levels by ELISA. Data presented indicates the mean pg/mL values and fold change as normalised to vehicle control, +/−SEM, n=4. Data were analysed by RM two-way ANOVA with Dunnett's post-test comparing each peptide concentration with vehicle or where normalised to lowest test peptide dose, **p<0.01, ****p<0.0001. (g), (h) Stimulated PBMCs were cultured together with the peptide IK14004 over a 5-concentration range plus vehicle control (0-1.25 μM) for 72 hrs after which supernatants were collected and assessed for IL-12p40 by ELISA. Data presented indicates the mean pg/mL values and fold change data (as normalised to vehicle control), +/−SEM, n=4. Data were analysed by RM two-way ANOVA with Dunnett's post-test comparing each peptide concentration with vehicle or where data were normalised peptide concentration was compared against lowest test peptide concentration, *p<0.05, **p<0.01, ***p<0.001. Dotted line indicates mean vehicle control for normalised data. (i) Effects of peptide on IL-12p70 production in a stimulated PBMC assay (24 hrs). PBMCs were cultured together with anti-CD3 (1 μg/ml) stimulation and Inter K peptides over a 5-concentration range (0-1.25 μM) for 24 hrs after which supernatants were collected and analysed for IL-12p70 by ELISA. Data presented indicates the mean cytokine production (pg/mL) in response to peptide treatment, +/−SEM, n=4. No statistical significance as determined by two-way ANOVA with Dunnett's post-test. (j), (k) IFN-γ expression in CD4+ and CD8+ T cells from stimulated PBMCs. Freshly isolated PBMCs were stimulated with (−aCD3) anti-CD3 (1 μg/mL) for 24 hrs in the presence of test peptide IK14004 at indicated concentrations (pM). Data are presented as mean+/−SEM from 4 donors. **p<0.01, ****p<0.0001, two-way ANOVA with Dunnett's post-test was used to compare concentrations of each peptide to vehicle (0). (l) Stimulated PBMCs were cultured together with peptide IK14004, over a 5-concentration range plus vehicle control (0-1.25 μM) for 72 hrs after which supernatants were collected and assessed for IFN-γ by ELISA. Data presented indicates the mean pg/mL values, +/−SEM, n=4. Data were analysed by RM two-way ANOVA with Dunnett's post-test comparing each peptide concentration with vehicle, **p<0.01, ****p<0.0001. (m) Isolated CD3+ T cells from PBMCs were cultured in the presence of anti-CD3/CD28 activation beads at a 4:1 T cell to bead ratio together with test peptide (0.08-1.25 μM) formulated in MQ water or vehicle control. After 72 hrs, supernatants were collected and assessed for IFN-γ by ELISA. Data presented as pg/mL+/−SEM, n=4; No statistical significance as determined using two-way ANOVA with Dunnet's post-test comparing peptide to vehicle control. (n) Immature monocyte derived DCs (iMoDCs) were derived from isolated CD14+ monocytes cultured for 7 days in Mo-DC differentiation media. iMoDCs were cultured for 72 hrs in the presence of test peptides over a 5-point concentration curve plus vehicle (0-1.25 μM) and anti-CD3 (1 μg/mL). After 72 hrs, cells were assessed for CD14 and CD11c expression by flow cytometry to determine DC cell phenotype. Data presented indicates the mean respective CD14 positive or negative cell populations (%) within CD11c positive population in response to peptide treatment, +/−SEM, n=4. Data were analysed by RM two-way ANOVA with Dunnett's post-test comparing each peptide concentration with vehicle, *p<0.05, ****p<0.0001. (o) PBMCs were cultured for 24 hrs together with vehicle control or test peptide (0.08-1.25 μM) and stimulated with soluble anti-CD3 (1 μg/mL). After 24 hrs, supernatant was collected and assessed for IL-10 concentration by ELISA. Data presented as pg/mL cytokine concentration+/−SEM, n=4. No statistical significance as determined using one-way ANOVA with Dunnett's post-test comparing peptide to the vehicle control. Red dotted line indicates stimulated media control only, blue dotted line indicates unstimulated control.

FIG. 4: Selective targeting of JAK/STAT signalling pathways by RSKAKNPLYR-(2Adod)₄-NH₂. (a) Human PBMCs (n=4, taken from normal healthy volunteers) were prepared from buffy coats using density gradient separation. CD3+ total T cell populations were isolated by immunomagnetic separation (StemCell, Cat #19051, Lot #19E102876A). Cells were resuspended in RPMI-10 (RPMI-1640 supplemented with 10% heat inactivated FBS, 100 U/mL penicillin, 100 μg/mL streptomycin, 2 mM L-glutamine, and 50 μM β-mercaptoethanol) at 0.5×10⁶/mL and plated at a density of 0.5×10⁵ per well (100 μL) in 96-well, flat bottom culture plates. Cells cultures were stimulated with anti-CD3/anti-CD28 coated Dynabeads (ThermoFisher, Cat #11131 D, Lot #00984668) at a 4:1 cell to bead ratio and cultured in the presence of peptides for 72 hrs. At the end of culture, cells were recovered and fixed using the BD Phosflow™ Fix buffer I (BD Bioscience, Cat #557870) and permeabilised to allow for intracellular staining using BD Phosflow™ Perm Buffer III (BD Bioscience, Cat. #558050). Cells were then stained with fluorochrome conjugated antibodies detecting phospho-STAT1 protein (PE Mouse Anti-Stat1 (pY701) #562069 (BD Bioscience, Lot #0170543)). Phospho-STAT1 intracellular expression was determined within the individual T cell populations by flow cytometry (a) PBMCs were used to isolate CD3+ T cells and isolated T cells were cultured for 72 hrs with vehicle control or test peptide (0.08-1.25 μM) and stimulated with anti-CD3/anti-CD28 Dynabeads™ at a 4:1 cell to bead ratio or left unstimulated. After 72 hours, cells were recovered and stained for CD3, CD4, CD8 and pSTAT1, and expression assessed using flow cytometry. Data shown as % CD4+ T cells (A), % pSTAT1 expression within CD4+ T cell populations (B) and pSTAT1 MFI (C)+/−SEM, n=4. *p<0.05, as determined using two-way ANOVA with Dunnett's post-test comparing peptide to the vehicle control. Red dotted line indicates stim only and blue dotted line indicates unstimulated control. Isotype control values for pSTAT1-PE: 345.25 MFI. (c) PBMCs recovered from buffy coat samples (n=4) were used to isolate CD3+, CD4+ T cell populations by immunomagnetic separation (StemCell, Cat. 19051C, 17852C, 17953C, respectively). Cells were resuspended in RPMI-10 at 0.5×106/mL and plated at a density of 0.5×105 per well (100 μL) in 96-well, flat bottom culture plates. Cells were stimulated with anti-CD3/anti-CD28 Dynabeads (ThermoFisher, Cat. 00788901) at a 4:1 cell to bead ratio and cultured for 72 hrs in the presence of test peptide IK14004, formulated in MQ water (Lot. 2152901). After 72 hrs, cells were recovered from isolated CD3+ T cell cultures and assessed for intracellular phosphoSTAT6 expression within the CD4+ T cells fraction by flow cytometry. Data presented as mean fluorescent intensity (MFI)+/−SEM, n=4; **p<0.01, ****p<0.0001, determined using two-way ANOVA with Dunnett's post-test comparing Inter-K peptide to vehicle control. (e) Isolated CD3+ T cells were cultured for 72 hrs together with vehicle control (0.13% H₂O), or IK14004 (0.08-1.25 μM), and stimulated with soluble anti-CD3 anti-CD28 stimulation beads (4:1 cell to bead ratio). After 72 hrs, cells were recovered and stained for pSTAT3 and expression assessed by flow cytometry. Data presented pSTAT3 expression (mean fluorescent intensity, MFI) CD4+ T cells, +/−SEM, n=4. No statistical significance as determined using a repeated measures (RM) two-way ANOVA with Dunnett's post-test comparing peptide to the vehicle control. The red dotted line represents the mean stimulated media only control value and the blue dotted line represents the mean unstimulated control group value. (f), (g) PBMCs were cultured for 72 hrs together with vehicle control (0.13% H₂O), or IK14004 (0.08-1.25 μM), and stimulated with soluble anti-CD3 (1 μg/mL). After 72 hrs, cells were recovered and stained for IL-6R (CD126) and expression assessed by flow cytometry. Data presented as CD126 expression (mean fluorescent intensity, MFI) within CD4+ T cells and CD8+ T cells, +/−SEM, n=4. *p<0.05, **p<0.01, ***p<0.001 as determined using a repeated measures (RM) two-way ANOVA with Dunnett's post-test comparing peptide to the vehicle control. The red dotted line represents the mean stimulated media only control value and the blue dotted line represents the mean unstimulated control group value.

FIG. 5: RSKAKNPLYR-(2Adod)₂-NH₂, like RSKAKNPLYR-(2Adod)₄-NH₂, activates Lck and inhibits c-Src.

FIG. 6: RSKAKNPLYR-(2Adod)₄-NH₂ inhibits MAP4K1 kinase activity.

FIG. 7: RSKAKNPLYR-(2Adod)₄-NH₂ increases CD28 levels on T cells. PBMCs were cultured together with anti-CD3 (1 μg/ml) stimulation and IK14004 over a 5-concentration range (0-1.25 μM) for 72 hrs after which cells were assessed for expression of CD28 by flow cytometry. Data presented indicates the mean respective expression in CD4+ T cell populations in response to peptide treatment, +/−SEM, n=4. Data was analysed by two-way ANOVA with Dunnett's post test, *P<0.05, **P<0.01, ***P<0.001, ****P<0.0001. *** indicates all bars under the line are significant compared to vehicle. Red dotted line indicates unstimulated PBMC expression.

FIG. 8: rskaknplyr-(2Adod)₄-NH₂ (RSKAKNPLYR-(2Adod)₄-NH₂ comprising D amino acids) increases IL-2 expression on exhausted CD4+ cells upon restimulation. Supernatants were collected after 72 hrs in culture to assess IL-2 cytokine production measured by multiplex immunoassay. Data are presented as mean+/−SEM from 4 biological replicates normalised to vehicle control (0). **P<0.01, Non-parametric one way ANOVA (Freidman) with Dunns post-test was used to compare groups at each dose level to lowest level (0.08 μM).

FIG. 9: RSKAKNPLYR-(2Adod)₄-NH₂ increases the proportion of Foxp3 expressing CD25+ cells.

FIG. 10: RSKAKNPLYR-(2Adod)₄-NH₂ increases the ratio of Tregs to CD4+ cells. The increased proportion of Foxp3 expressing CD25+ cells is reflected in the CD4/Treg ratio at higher concentrations of RSKAKNPLYR-(2Adod)₄-NH₂.

FIG. 11: RSKAKNPLYR-(2Adod)₄-NH₂ induced increase in the proportion Foxp3 expressing CD25+ cells is not associated with a statistically significant increase in the level of Foxp3 expression.

FIG. 12 shows intraperitoneal administration of RSKAKNPLYR-(2Adod)₄-NH₂ (“IK14004”) reduces tumour area in the lungs in a Lewis Lung Cancer (LCC) metastasis model. RSKAKNPLYR-(2Adod)₄-NH₂ was administered intraperitoneally (400 μg) twice per week for two weeks after which H&E sections were assessed for evidence of tumour infiltration and tumour mass was calculated as percentage of healthy lung tissue. Data points show the mean area of tumour mass within the lung per sample. n=16, **p<0.01, unpaired two-tailed t-test.

FIG. 13 shows RSKAKNPLYR-(2Adod)₄-NH₂ (“IK14004”) reduces xenograft tumour volume and tumour cell viability, in a Lewis Lung Cancer (LLC) xenograft model.

FIG. 14 shows a) RSKAKNPLYR-(2Adod)₄-NH₂ (“IK14004”) does not inhibit B16F10 melanoma cell proliferation, b) RSKAKNPLYR-(2Adod)₄-NH₂ (“IK14004”) does not inhibit Lewis Lung Cancer cell proliferation.

FIG. 15 shows RSKAKNPLYR-(2Adod)₄-NH₂ (“IK14004”) reduces lung nodules, in metastatic lung cancer model.

FIG. 16 shows RSKAKNPLYR-(2Adod)₄-NH₂ (“IK14004”) enhances IL-12 receptor expression on NK cells.

FIG. 17 shows RSKAKNPLYR-(2Adod)₄-NH₂ (“IK14004”) enhances IL-12 receptor expression on NK cells.

FIG. 18 shows RSKAKNPLYR-(2Adod)₄-NH₂ (“IK14004”) enhances NKp44 expression on NK cells.

FIG. 19 shows RSKAKNPLYR-(2Adod)₄-NH₂ (“IK14004”) enhances NKG2D receptor expression on NK cells.

### DETAILED DESCRIPTION

Members of the non-receptor Src kinase family (SKF) include the following eight kinases in mammals: Src, Fyn, Yes, Fgr, Lyn, Hck, Lck, and Blk.

The present invention is based in part on the development of a synthetic peptide that has opposing effects on the activities of c-Scr and Lck, and which allows maintenance of homeostatic levels of IL-2.

In particular, the present inventors demonstrate in Example 1 that RSKAKNPLYR-(2Adod)₄-NH₂ and RSKAKNPLYR-(2Adod)₂-NH₂ inhibits c-Src and activates Lck.

Accordingly, in one embodiment the present invention provides a method of treating or preventing an autoimmune disorder in a subject, said method comprising administering to the subject a therapeutically effective amount of a peptide comprising an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂.

As used herein RSKAKNPLYR-(2Adod)₁-NH₂ is referred to interchangeably as IK14001 or RSKAKNPLYR-(2Adod1). As used herein RSKAKNPLYR-(2Adod)₂-NH₂ is referred to interchangeably as IK14002 or RSKAKNPLYR-(2Adod2). As used herein RSKAKNPLYR-(2Adod)₃-NH₂ is referred to interchangeably as IK14003 or RSKAKNPLYR-(2Adod3). As used herein RSKAKNPLYR-(2Adod)₄-NH₂ is referred to interchangeably as RSKAKNPLYR-(2Adod4) or IK14004.

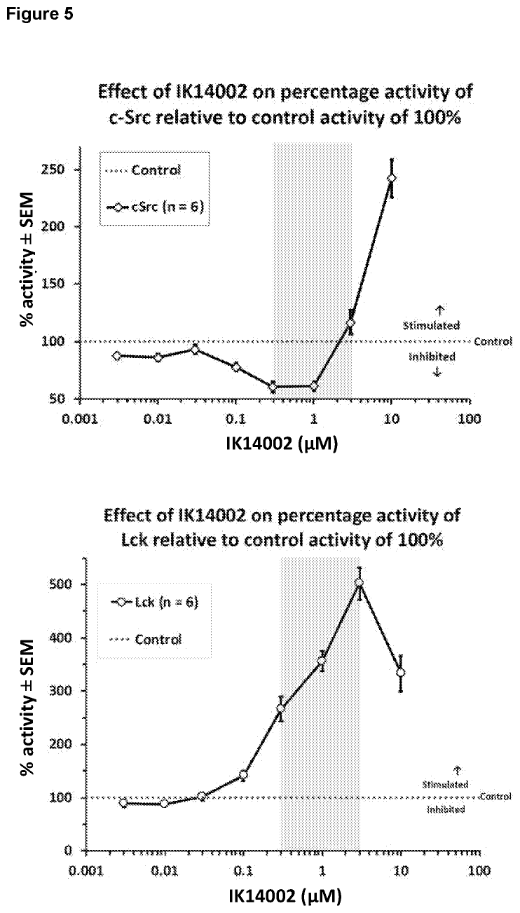

The data of Example 1 suggest that RSKAKNPLYR-(2Adod)₄-NH₂ exerts a degree of selectivity given its effects on Csk and SFK members. The present inventors have also demonstrated in FIG. 5 demonstrates that RSKAKNPLYR-(2Adod)₂-NH₂, like RSKAKNPLYR-(2Adod)₄-NH₂, inhibits c-Src and activates Lck, and that RSKAKNPLYR-(2Adod)₂-NH₂ behaves similarly to RSKAKNPLYR-(2Adod)₄-NH₂ at 1 and 3 uM. (data not shown).

This result is surprising in part because the Src kinase family members are non-receptor tyrosine kinases that share a similar structure which presents a challenge since activation and inactivation via conserved tyrosines is likely to be a shared feature amongst SFKs. Hence, all Lck activators reported to date have been shown to activate other SFK members. Moreover, given the non-selectivity of SFK inhibitors together with the presence of multiple SFKs expressed in immune cells, it remains difficult to implicate any given member of the SFK family in a particular signalling pathway, for example, to allow modulation of a desired signalling pathway.

As used herein, the term “activating” generally refers to increasing an activity of at least one target protein. In the specific context of a kinase this activation leads to increased phosphorylation of at least one target substrate or site. This activation can be caused by any means including (but not limited to) increasing the probability that a complex forms between a protein kinase and a binding partner of the protein kinase, or increasing the activity of the kinase once bound to its target. Such activation may take place either in vivo or in vitro.

As used herein, the term “inhibiting” generally refers to decreasing an activity of at least one target protein. This inhibition can be caused by any means including (but not limited to) decreasing the probability that a complex forms between a protein kinase and a binding partner of the protein kinase, or decreasing the activity of the kinase once bound to its target. Such inhibition may take place either in vivo or in vitro.

As used herein the term “treating” includes therapeutic treatment as well as prophylactic treatment (either preventing the onset of a disorder or a symptom of a disorder altogether or delaying the onset of a symptom of a disorder, or a preclinically evident stage of a disorder in an individual.

The term “preventing” includes either preventing the onset of a disorder or a symptom of a disorder altogether or delaying the onset of disorder or a symptom of a disorder, or a preclinically evident stage of a disorder in an individual. This includes prophylactic treatment of those at risk of developing a disease, such as an autoimmune disease, for example. “Prophylaxis” is another term for prevention.

As used herein the term “subject” includes human and hon-human subjects. Preferably, the subject is a mammal.

As used herein “autoimmune disorder” refers to a disease arising from an overactive immune response of the body against substances and tissues normally present in the body, e.g. an inflammatory condition. The terms autoimmune disease and autoimmune disorder are used interchangeably herein.

The term “effective amount” (for example a “therapeutically effective amount” or a “pharmaceutically effective amount”) as used herein refers to an amount of a peptide as described herein that results in the desired molecular or cellular response, for example, IL-2 homeostasis. Said “effective amount” will vary from subject to subject, depending on the age and general condition of the individual and with the factors such as the particular autoimmune condition being treated or prevented, the duration of the treatment, previous treatments and the nature and pre-existing duration of the autoimmune condition. An effective amount of a peptide includes an amount that can be administered to a subject without excessive or non-tolerable toxicity, irritation, allergic response, or other problem or complication, commensurate with a reasonable benefit/risk ratio, but one that is sufficient to provide the desired effect as assessed by an appropriate technique such as those disclosed throughout this specification. Thus, while it is not possible to specify an exact effective amount, those skilled in the art will be able to determine an appropriate “effective” amount in any individual case using routine experimentation and background general knowledge. A therapeutic result in this context includes eradication or lessening of symptom. A therapeutic result need not be a complete amelioration of the condition (i.e. a cure).

A peptide as described herein can be administered to the mammal in accordance with a method of the invention, or cells can be contacted with the peptide in vitro. Likewise, the invention provides for ex vivo treatment where cells are treated with the peptide externally of the subject prior to return, administration to, or implantation of the cells in, the subject.

A peptide as described herein can be provided in a pharmaceutical composition comprising a pharmaceutically acceptable carrier and/or excipient for administration to the intended subject. The peptide can be administered orally, intranasally, via inhalation (e.g., by aerosol spray), intravenously, parenterally, rectally, subcutaneously, by infusion, topically, intramuscularly, intraperitoneally, intraspinally, intraocularly, or via any other route deemed appropriate, including administration into a joint of a subject.

Preferably, the peptide is administered orally and/or topically.

In one embodiment, topical administration involves administration into a joint.

A pharmaceutical composition can, for example, be in the form of a liquid, suspension, emulsion, syrup, cream, ingestable tablet, capsule, pill, suppository, powder, troche, elixir, or other form that is appropriate for the selected route of administration.

Pharmaceutical compositions useful in methods in accordance with the invention include aqueous pharmaceutical solutions. Injectable compositions will be fluid to the extent that syringability exists and typically, will normally be stable for a predetermined period to provide for storage after manufacture. Moreover, a pharmaceutically acceptable carrier may include any suitable conventionally known solvents, dispersion media, water, physiological saline and isotonic preparations or solutions, surfactants, and any suitable pharmaceutically acceptable carrier (e.g., orally or topically acceptable carriers) may be utilised. Suitable dispersion media can for example contain one or more of ethanol, polyols (e.g., glycerol, propylene glycol, liquid polyethylene glycol and the like), vegetable oils and mixtures thereof. In particular, the Lck modulator described herein or nucleic acid can, for example, be formulated with an inert diluent, an assimilable edible carrier and/or it may be enclosed in a hard or soft shell gelatin capsule.

A pharmaceutical composition as described herein can also incorporate one or more preservatives suitable for in vivo and/or topical administration such as parabens, chlorobutanol, phenol, sorbic acid, and thimerosal. In addition, prolonged absorption of the composition may be brought about by the use in the compositions of agents for delaying absorption such as aluminium monosterate and gelatin. Tablets, troches, pills, capsules and the like containing a peptide as described herein can also contain one or more of the following: a binder such as gum tragacanth, acacia, corn starch or gelatin; a disintegrating agent such as corn starch, potato starch or alginic acid; a lubricant such as magnesium stearate; a sweetening agent such as sucrose, lactose or saccharin; and a flavouring agent.

The use of ingredients and media as described above in pharmaceutical compositions is well known. Except insofar as any conventional media or ingredient is incompatible with a Lck modulator as described herein, use thereof in therapeutic and prophylactic pharmaceutical compositions as described herein is included.

By “combination therapy” as used herein is meant prior, simultaneous or sequential administration of the peptide in accordance with the invention in the same or different formulations to the other drug(s) by the same or different routes whereby the Lck modulator(s) and and/or nucleic acid(s) exert their effect(s) in over overlapping therapeutic windows.

It is particularly preferred to formulate parenteral compositions in dosage unit form for ease of administration and uniformity of dosage. Dosage unit form as used herein is to be taken to mean a physically discrete unit suited as a unitary dosages for the subject to be treated, each unit containing a predetermined quantity of at least one Lck modulator or nucleic acid in accordance with the invention calculated to produce the desired therapeutic or prophylactic effect in association with the relevant carrier and/or excipient used. When the dosage unit form is for example, a capsule, tablet or pill, various ingredients may be used as coatings (e.g., shellac, sugars or both) to otherwise modify the physical form of the dosage unit or to facilitate administration to the subject.

A pharmaceutical composition will generally contain at least about 1% by weight of a peptide as described herein. The percentage may be varied and can conveniently be between about 5% to about 80% w/w of the composition or preparation. Again, the amount of a peptide in accordance with the invention will be such that a suitable effective dosage will be delivered to the subject taking into account the proposed route of administration. Preferred oral pharmaceutical compositions will contain between about 0.1 μg and 15 g of the peptide.

The dosage of the Lck modulator or nucleic acid in accordance with the invention will depend on a number of factors including whether the peptide is to be administered for prophylactic or therapeutic use, the disease, condition or purpose for which the agent is intended to be administered, the severity of the disease or condition, the age of the subject, and related factors including weight and general health of the subject as may be determined by the physician or attendant in accordance with accepted principles. For instance, a low dosage may initially be given which is subsequently increased at each administration following evaluation of the subject's response. Similarly, the frequency of administration may be determined in the same way that is, by continuously monitoring the subject's response between each dosage and if necessary, increasing the frequency of administration or alternatively, reducing the frequency of administration.

Typically, a peptide as described herein will be administered in accordance with a method embodied by the invention to provide a dosage of the peptide of up to about 100 mg/kg body weight of the individual, more usually in a range up to about 50 mg/kg body weight, and most usually in a range of about 5 mg/kg to 40 mg/kg body weight. In at least some embodiments, the peptide will be administered to provide a dosage of the peptide in a range of from about 5 to 25 mg/kg body weight, usually in a range of from about 5 mg/kg to about 20 mg/kg and more usually, in a range of from 10 mg/kg to about 20 mg/kg. When administered orally, up to about 20 g of the peptide may be administered per day, (e.g., 4 oral doses per day, each dose comprising 5 g of the peptide).

With respect to intravenous routes, particularly suitable routes are via injection for systemic distribution of the peptide into blood vessels which supply tissue or particular organ(s) to be treated. Moreover, the peptide can be delivered by any suitable infusion or perfusion techniques. The peptide may also be delivered into cavities such for example the pleural or peritoneal cavity, or be injected directly into tissue to be treated.

In some embodiments, the sequence is an isolated or purified sequence.

Methods of “isolation” and “purification” of a sequence produced by natural or recombinant techniques are known in the art for example in C-H Lee, A Simple Outline of Methods for Protein Isolation and Purification, Endocrinology and Metabolism; 2017, March; 32(1): 18. Further, the terms “isolated” or “purified” include synthesised and other artificially produced sequences. Methods for synthesising sequences are known in the art. Generally, sequences are chemically synthesized by the condensation reaction of the carboxyl group of one amino acid to the amino group of another. Chemical synthesis of sequences can be carried out using solution-phase techniques or solid-phase techniques. Synthetic techniques can allow for the production of sequences incorporating unnatural amino acid sequences, backbone modification and synthesis of D-isomers.

As described herein, the present inventors have identified that the Lck modulating polypeptides described herein can comprise L or D amino acids and have biological activity, e.g. in inducing low levels of IL-2. As is known in the art, alpha amino acids include a chiral carbon at the alpha position. Consequently, all alpha amino acids, with the exclusion of glycine can exist in either of two enantiomers, being the L- or D-isomers. Generally, only L-amino acids are manufactured in mammalian cells and incorporated into proteins. D-amino acids can be artificially synthesised or may be found in bacterial proteins. The L and D convention is not used to directly refer to the stereochemistry of the amino acids, rather it is used in reference to amino acid configuration and does not refer to the optical activity of the amino acid itself, but rather to the optical activity of the isomer of glyceraldehyde from which that amino acid can be synthesized (D-glyceraldehyde is dextrorotatory; L-glyceraldehyde is levorotatory).

As used herein, lowercase indicates dextrorotatory (“dextro”) amino acids, and so the peptide rskaknplyr-(2Adod)₄-NH₂ comprises D amino acids.

In one embodiment, the peptide comprises the amino acid sequence rskaknplyr-(2Adod)₂-NH₂ or rskaknplyr-(2Adod)₄-NH₂

In some embodiments, the sequences of the invention are modified. In some embodiments, the modification may be a modification that alters the pharmacological properties of the sequences. In some embodiments, the modification increases the half-life of the composition or sequences of the invention. In some embodiments, the modification may increase the bioactivity of the sequences (and/or the composition of the invention). In some embodiments, the modification may be a modification that increases selectivity of the sequences or compositions of the invention.

In one embodiment, the modification is the addition of a protecting group. The protecting group may be an N-terminal protecting group, a C-terminal protecting group or a side-chain protecting group. The sequences of the present invention may have one or more of these protecting groups. The person skilled in the art is aware of suitable techniques to react amino acids with these protecting groups. These groups can be added by preparation methods known in the art. The groups may remain on the sequences or may be removed prior to use or administration. The protecting group may be added during synthesis.

The present inventors have demonstrated that amidating the Lck activating peptide surprisingly increases the level of Lck activity. Accordingly, in one embodiment the present invention provides a peptide as described herein, wherein the distal most fatty acid is amidated.

As used herein in context of a polypeptide sequence, “NH₂” indicates the polypeptide is amidated.

In some embodiments, the sequence is amidated at its C-terminus. Amidation refers to the process of N-oxidative cleavage of glycine-extended substrates by sequential endo- and exoproteolysis. Methods are known in the art for producing amidated sequences in vitro, such as: enzymatic amidation; chemical modification of the C-terminus of recombinantly produced sequences and proteins; use of amide resins in solid-phase sequences synthesis; use of carboxypeptidase in the presence of ammonia; and conversion of the C-terminus of sequences to the methyl ester and addition of ammonia at low temperature. Examples of the disclosure of suitable techniques include D J Merkler, C-terminal amidated sequences: production by the in vitro enzymatic amidation of glycine-extended sequences and the importance of the amide to bioactivity; Enzyme Microbial technology, 1994, June; 16(6): 450-6 and V Čeřovský and M-R Kula C-Terminal sequences Amidation Catalyzed by Orange Flavedo sequences Amidase; Angewandte Chemie, 1998, August; 37(13-14): 1885.

Amidation of the C-terminus results in the C-terminal end being uncharged, so the modified sequences more closely mimic a native protein. This can have a series of advantages including an enhanced ability of the sequence to enter a cell; an improvement in the metabolic stability of the sequence in vivo; a decrease in the in vivo enzymatic degradation of the sequences by aminopeptidases, exopeptidases, and synthetases; and an improvement of the shelf-life of the sequences.

As used herein in context of a polypeptide sequence, the term Adod refers to aminododecanoic acid, and “2Adod” refers to 2-amino dodecanoic acid; Where more than one fatty acid is coupled the number of fatty acids coupled is denoted by a subscript. For example, “(2Adod)₂” denotes two 2-aminododecanoic acids. Thus, a single unit of a polyamide moiety as described herein, corresponds to one 2-amino dodecanoic acid residue (also referred to herein as “(2Adod)₁”. Two units of a polyamide moiety as described herein, corresponds to two 2-amino dodecanoic acid residues (also referred to herein as “(2Adod)₂”. Three units of a polyamide moiety as described herein, corresponds to three 2-amino dodecanoic acid residues “(2Adod)₃”. Four units of a polyamide moiety as described herein, corresponds to four 2-amino dodecanoic acid residues “(2Adod)₄”.

The present inventors have demonstrated in Example 1 that that the coupling of two or four fatty acids to the peptide RSKAKNPLYR, confer on the peptide an increased ability inhibit c-Src activity while also activating Lck activity, in contrast to coupling of one or three fatty acids to the peptide RSKAKNPLYR, which did not inhibit c-Src.

While inhibition of Lck kinase has been proposed as treatment against both autoimmunity and cancer (Bommhardt U et al, Int J Mol Sci, 2019, 20(14): 3500) the role of Lck in some solid cancers remains controversial. For example, Lck is over-expressed in NSCLC cell lines (Ripniewska E et al, Oncotarget, 2018, 9: 27346-27362) yet its expression in tumour infiltrates of lung cancer patients is associated with a good prognosis (D'Andrilli A et al, Interactive Cardiovascular & Thoracic Surgery, 2012, 15: 148-151). Moreover, genomic analysis of melanomas has shown that Lck expression is associated with significantly improved survival (Cancer Genomic Atlas Network, Cell, 2015, 161: 1681-96). Based on the data presented herein, the present inventors propose that the main role of Lck in solid cancers is directing positive therapeutic outcomes guided by appropriate immune responses, rather than intrinsic cancer cell Lck abnormalities (Creeden J F et al, Int J Mol Sci, 2020, 21: 8823). Furthermore, the anti-tumour activity mediated by NK cells described herein is regulated by cytokine receptor signalling pathways which in many instances involve Lck. For example, IL-2-mediated activation of NK cells leads to activation of NK immunoglobulin-like receptors such as NKG2D (Konjevic G et al, Melanoma Res, 2010, 20(6): 459-67; Le Bert N et al, Immunol Cell Biol, 2014, 92: 230-6; Hu W et al, Front Immunol, 2019, doi.org/10.3389/fimmu.2019.01205; Skak K et al, Immunology, 2008, 123(4): 575-583) and Lck is central to downstream signalling from NKG2D associated with enhanced cytotoxicity ((Rajasekaran R et al, Front Immunol, 2016, doi.org/10.3389/fimmu.2016.00176).

In some embodiments, the peptide comprises two or four linked fatty acid moiety(ies).

In a preferred embodiment, the present invention provides a peptide comprising an amino acid sequence selected from the group consisting of RSKAKNPLYR-(2Adod)₄ and RSKAKNPLYR-(2Adod)₂.

In another preferred embodiment, the present invention provides a peptide consisting of an amino acid sequence selected from the group consisting of RSKAKNPLYR-(2Adod)₄ and RSKAKNPLYR-(2Adod)₂.

The coupled 2Adod may be provided by coupling together the fatty acids by consecutively forming a respective amide bond between the amino group substituent of one fatty acid chain and the terminal carboxyl group of the next fatty acid to thereby provide coupled fatty acids.

Accordingly, fatty acids having an amino group (NH₂) substituent on the a or p carbon of the fatty acids are particularly suitable for coupling.

In another preferred embodiment, the present invention provides a peptide for activating Lck as described herein, wherein the distal most fatty acid is amidated.

The present inventors have demonstrated in Example 2 that RSKAKNPLYR-(2Adod)₄-NH₂ activates IL-2 production, and in Example 5 that rskaknplyr-(2Adod)₄-NH₂ activates IL-2 production.

Importantly, maintenance of a low-level increase in IL-2 levels was observed, and high levels of IL-2 are avoided.

Accordingly, in one embodiment the present invention provides a method as described herein, wherein the autoimmune disorder is a disorder associated with dysregulated IL-2 homeostasis.

The major role of IL-2 during homeostasis and activation of the immune system is well recognised. Under steady state conditions, low levels of IL-2 are maintained primarily by activated CD4+ T cells and in secondary lymphoid organs IL-2 is consumed at the same site by immune-suppressive T regulatory cell populations (Tregs). IL-2 is also produced by dendritic cells (DCs) and activated DCs express CD25 on their cell surface for binding to either T-cell- or DC-derived IL-2 presented in trans to adjacent effector cells. Hence, during an immune response the IL-2 produced by activated CD4+ and CD8+ T cells is consumed by DCs, Tregs, and CD25+ effector CD4+/CD8+ cells with IL-2 signals controlling the expansion of antigen-specific CD8+ T cell populations.

Importantly, only low IL-2 levels are required to maintain immunosuppressive T cell populations that counter chronic autoimmune conditions given that Tregs are exquisitely dependent on IL-2 for survival. Moreover, humans deficient in IL-2 and the high-affinity IL-2Ralpha receptor chain (CD25) develop systemic autoimmunity due to impaired production of CD4+/CD25+ immunosuppressive Tregs and low-dose IL-2 therapy for SLE, graft-versus-host disease, and type I diabetes has yielded encouraging results. Subcutaneous injection of low-dose IL-2 in patients with SLE results in decreases in the SLE Disease Activity Index (SLEDAI); however, the very short half-life of IL-2 remains a challenge.

The selective enhancement of Lck activity by IK14004 and fine-tuning of IL-2 production (e.g. within a therapeutic window of IL-2), combined with suppression of Th1-skewed cytokines described herein is relevant in the treatment and/or prevention of autoimmune disorders. The role of IL-2 during homeostasis of the immune system is well recognised and immune-suppressive T regulatory (Treg) cells are critical in maintaining immune tolerance. Targeted deletion of Tregs in mice leads to severe autoimmunity and production of Tregs is enhanced by TCR activation with anti-CD3/CD28 antibodies. Under steady state conditions, low levels of IL-2 are maintained primarily by activated CD4+ T cells and consumed in secondary lymphoid organs by Tregs. The use of low-dose IL-2 to prevent autoimmunity (in contrast to the use high-dose IL-2 to expand cytotoxic lymphocyte populations) accords with clinical evidence in so far as humans deficient in IL-2 and CD25 develop systemic autoimmunity and low-dose IL-2 therapy for SLE, graft-versus-host disease has yielded encouraging results.

Notwithstanding the demonstration herein that IK14004 inhibits IFNg production, avoidance of excessive IL-2 production minimises the great risk from a downstream cascade of immune modulators such as IFNg produced by IL-2 responsive cells which then stimulate cytolytic mechanisms. For example, excessive IFNg signalling has been associated with auto-inflammatory diseases in mice and humans and elevated levels of IFNg in multiple sclerosis are thought to be due to IL-12 effects.

Without wishing to be bound by theory the present inventors propose, low dose IL-2 immunotherapy has been proposed to maintain Treg populations and treat autoimmunity/chronic inflammatory conditions/tissue graft rejection, while high dose IL-2 administration be used to expand cytotoxic lymphocyte populations and which is to be avoided for the treatment of autoimmune disorders.

The present inventors have also demonstrated in Example 1 that Lyn is not inhibited. Lyn tyrosine kinase regulates inhibitory signaling in B and myeloid cells—loss of Lyn results in a lupus-like autoimmune disease with hyperactive B cells and myeloproliferation, and maintaining Lyn avoids autoimmunity.

The present inventors have further demonstrated in Example 1 that Hck is inhibited. Hck inhibitors are also well known for their regulatory role in various malignancies and autoimmune diseases.

Importantly, Example 2 demonstrates that the peptide could also stimulate a Lck-independent signalling pathway.

In addition to the canonical Lck-regulated TCR signalling pathway, immune responses are also mediated via guanine nucleotide-binding proteins (G proteins) in both Lck-independent and Lck-dependent ways with fine-tuning of proximal TCR signals via a negative regulatory role for G proteins in transcriptional activation of cytokine responses.

Example 3 demonstrates RSKAKNPLYR-(2Adod)₄-NH₂ enhances expression of GNA11 on normal human T-cells.

In one embodiment the present invention provides a method as described herein, wherein the autoimmune disorder is a disorder associated with dysregulated IL-2 homeostasis. In one embodiment the dysregulated IL-2 homeostasis is inflammation.

As used herein, a disorder associated with dysregulated IL-2 homeostasis includes disorders that result from dysregulated IL-2 homeostasis in a subject. Without wishing to be bound by theory, IL-2 signal plays a major role in thymic treg differentiation, the homeostasis and function of treg cells, and it has been demonstrated that low-dose IL-2 is able to treat diseases through expansion of treg cells. The present inventors have demonstrated that the peptides described herein can induce low-dose IL-2, and avoid inducing high levels of IL-2. High dose IL-2 is associated with severe side effects and limited efficacy.

In one embodiment the disorder associated with dysregulated IL-2 homeostasis is a disorder that would benefit from low-dose IL-2 production and expansion of treg cells. For example, hematopoietic stem cell transplantation (HSCT), HCV-induced vasculitis, Type 1 diabetes, graft-versus-host disease (GVHD), alopecia areata and systemic lupus erythematosus (SLE), which would benefit from low-dose IL-2 production and expansion of treg cells.

Many autoimmune diseases are characterized by a decreased Tregs function or frequency, and may be regarded as proper pro-inflammatory “low Treg” chronic diseases. In contrast, advanced cancer is an anti-inflammatory, “high Treg” disease. For these reasons, autoimmune diseases and advanced cancer can be considered immunological opposites.

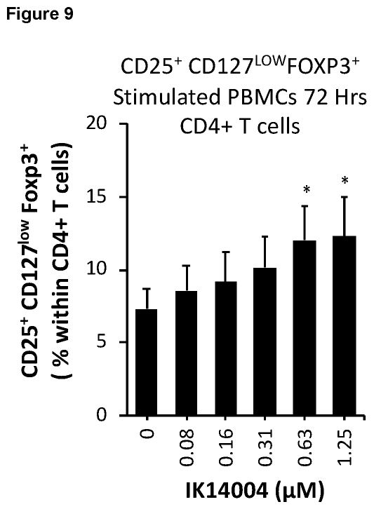

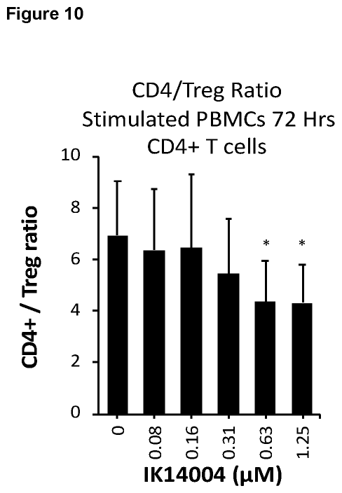

The present inventors have demonstrated in Example 6 that RSKAKNPLYR-(2Adod)₄-NH₂ increased the proportion of CD25-expressing cells that also expressed Foxp3 increased in the presence of IK14004 (FIG. 9) which was reflected in the CD4/Treg ratio at higher IK14004 concentrations (FIG. 10). However, this was not associated with a statistically significant increase in the level of Foxp3 expression (FIG. 11). Without wishing to be bound by theory, the present inventors propose that the peptides described herein can be used to promote levels of Tregs in a subject to treat and/or prevent autoimmunity in a subject with cancer, including subjects undergoing cancer therapy, including immunotherapy.

In a preferred embodiment, the Treg cells are Foxp3 positive cells.

In another embodiment, the Treg cells are CD25+ Foxp3+ cells.

In one embodiment the present invention provides a method as described herein, wherein the autoimmune disorder is an IL-2 mediated disorder.

As used herein, an IL-2 mediated disorder incudes disorders for which administration of IL-2, alone or in combination with other interventions (e.g., chemotherapy, immunotherapy, transplantation, etc.), is a bona fide treatment option and/or an investigational treatment option include but are not limited to cancers, systemic lupus erythematosus, rheumatoid arthritis, ankylosing spondylitis, psoriasis, Behcet's disease, Wegener's granulomatosis, Takayasu's disease, Crohn's disease, ulcerative colitis, autoimmune hepatitis, sclerosing cholangitis, Gougerot-Sjogren syndrome, alopecia areata, disorders requiring organ (e.g., liver, kidney, etc.) or tissue (e.g., bone marrow) transplantation, graft versus host disease (GVHD) and disorders treatable with stem cell transplantation (SCT) (including e.g., acute lymphoblastic leukemia, acute myelogenous leukemia, chronic myelogenous leukemia, myelodysplastic syndrome, myeloproliferative disorder, Hodgkin lymphoma, non-Hodgkin lymphoma, non-malignant diseases requiring allogeneic SCT, and the like), HIV Infection, Wiskott-Aldrich syndrome (WAS), X-linked thrombocytopenia, nephrotic syndrome, type 1 diabetes, macrophage activation syndrome, multiple sclerosis (including relapsing remitting), amyotrophic lateral sclerosis, etc.

In another aspect the present invention provides a method of treating an IL-2 mediated disorder in a subject in need thereof, the method comprising administering to a subject a composition comprising a peptide as described herein or a pharmaceutical composition as described herein simultaneously or sequentially with other interventions (e.g. immunotherapy).

In one embodiment the present invention provides a method as described herein, wherein the autoimmune disease is associated with dysregulated IL-2 and/or IL-2Ralpha (CD25) production.

Humans deficient in IL-2 and the high-affinity IL-2Ralpha receptor chain (CD25) develop systemic autoimmunity due to impaired production of CD4+/CD25+ immunosuppressive Tregs and low-dose IL-2 therapy for SLE, graft-versus-host disease, and type I diabetes has yielded encouraging results.

Accordingly, in one embodiment the present invention provides a method as described herein, wherein the subject is deficient in IL-2 and/or IL-2Ralpha (CD25) production.

Low dose IL-2 immunotherapy has been proposed to maintain Treg populations and treat autoimmunity/chronic inflammatory conditions/tissue graft rejection, while high dose IL-2 administration be used to expand cytotoxic lymphocyte populations.

In another embodiment the present invention provides a method as described herein, wherein the autoimmune disorder is selected from the group consisting of allergic asthma, rheumatoid arthritis, multiple sclerosis, systemic lupus erythematosis and other lupus disorders, Type 1 insulin-dependent diabetes mellitus (IDDM), psoriasis, scleroderma, glomerular nephritis, ankylosing spondylitis, and GVHD.

Importantly, consistent with the regulatory role for IK14004 in preventing excessive Th1 cytokine expression upon TCR-pMHC engagement by maintaining Lck-mediated phosphorylation of NFAT1, the peptide also inhibits the activity of CaMKIV as assessed in non-cell-based kinase profiling.

Activated Lck phosphorylates NFAT1 which serves to retain NFAT1 in the cytosol and thus prevent production of IL-2 and IFNg. The present inventors demonstrate in Example 4 that IL-12p40 production and IFNg production was inhibited from stimulated PBMC and isolated CD3+ T cell cultures.

IL-2 is used clinically to treat a number of human diseases including cancer, however the off-target effects of IL-2 has limited clinical therapies. Without wishing to be bound by theory, the present inventors propose that autoimmune disorders in cancer patients, including autoimmune effects in cancer patients during cancer therapy, can be treated using a peptide described herein. Accordingly, in one embodiment the present invention provides a method as described herein, wherein the subject has cancer. In one embodiment the present invention provides a method as described herein, wherein the effective amount of a peptide comprising RSKAKNPLYR-(2Adod)₂-NH₂ and/or RSKAKNPLYR-(2Adod)₄-NH₂ modulates the activity of Lck and/or G-protein signalling to maintain homeostatic levels of IL-2 in the subject. Prevention of tumour progression and immune-related adverse events (irAEs) secondary to immune checkpoint inhibitor (ICI) anti-cancer therapy rely on opposing immune responses. Patients with autoimmune disease have an increased risk of cancer (Valencia J C et al, J Interferon Cytokine Res, 2019, 39(1): 72-84) and both conditions commonly co-exist (Ma Q et al, BMC Cancer 18, Article number: 145 (2018). While significant improvements in outcome from cancer have occurred with introduction of immune checkpoint inhibitors (ICIs), autoimmunity is emerging as the nemesis of cancer therapy (Kumar P et al, Autoimmunity, 2018, 95: 77-99; Bakacs T et al, Scandinavian J Immunology, 2019, doi.org/10.1111/sji.12821; Lim S Y et al, Clin Cancer Res, 2019, doi: 10.1158/1078-0432.CCR-18-2795; Walsh S R et al, J Clin Invest, 2018, doi.org/101172/JC1121004).

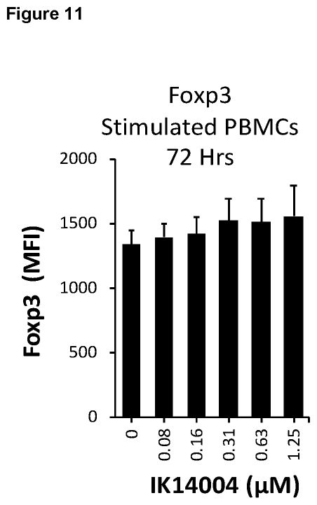

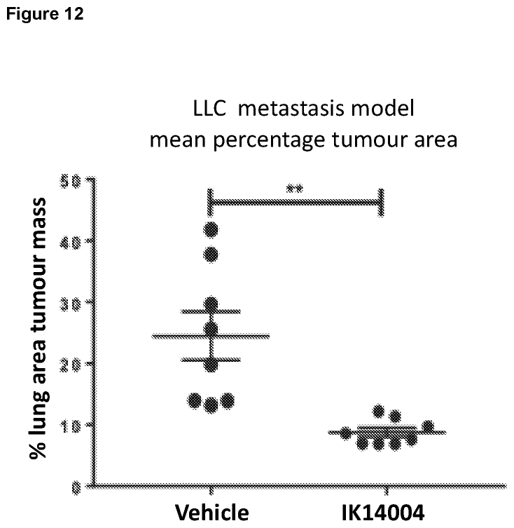

Without wishing to be bound by theory, the present inventors propose the uniqueness of IK14004 is that this lipidic peptide induces immune responses in two distinct lymphocyte populations which has the potential to address two contrasting unmet needs in health care; preventing cancer development and progression on the one hand while still maintaining immune responses that are needed to prevent autoimmunity. The present inventors propose that this arises because of distinct effects of IK14004 on T cells versus natural killer (NK) cells. Specifically, IK14004 demonstrates a unique property of simultaneously induced immune responses directed at suppression of autoimmunity, ie, enhancement of immunosuppressive T regulatory (Treg) CD4+/CD25+/Foxp3+ cell populations (e.g. FIG. 9) combined with induction of low but, importantly, not high-level secretion of IL-2 by T cells required to sustain Tregs whilst simultaneously suppressing production of pro-inflammatory cytokines such as IFN-g and IL-12 produced by T cells and dendritic cells (DCs), respectively. On the other hand, the anticancer effects of IK14004 (e.g. see FIGS. 12 to 15) are proposed to be mediated via its enhancement of receptor expression on NK cells required for anti-tumour cytotoxicity by NK cells, ie, enhanced IL-12 receptor expression that can respond to IL-12 produced by cancer cells which drives IFN-g production by NK cells as well as enhanced expression of natural cytotoxicity receptors.

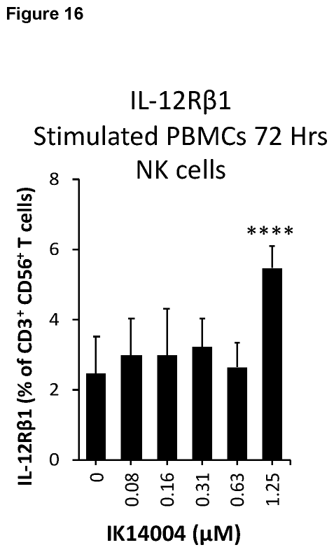

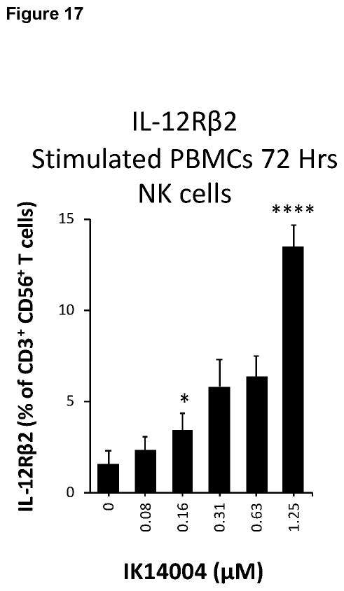

For example, FIGS. 16 and 17 demonstrate that RSKAKNPLYR-(2Adod)₄-NH₂ (“IK14004”) enhances IL-12 receptor expression on NK cells.

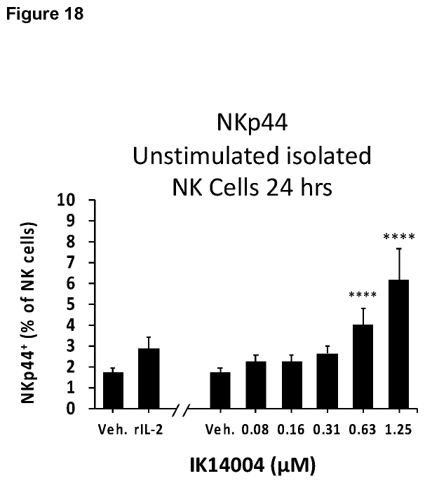

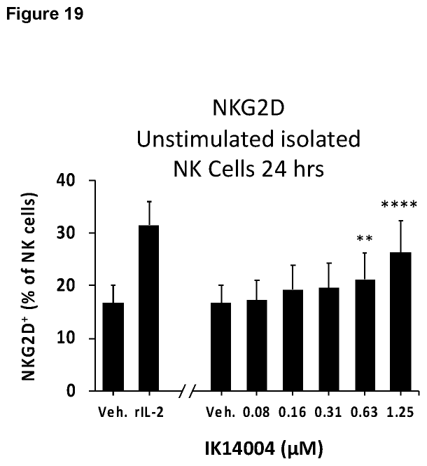

IL-2 has shown efficacy against cancer notwithstanding toxic side effects (Sun Z et al, Nat Communications 10, Article number: 3874 (2019) consistent with its known activation of NK cells (Sun Z et al, above; Hu W et al, Front Immunol, 2019, doi.org/10.3389/fimmu.2019.01205) that serves to suppress tumour growth (Spolski R et al, Nat Rev Immunol, 2018, 18: 648-659; Liao W et al, Immunity, 2013, 38(1): 13-25). An important role for IL-2 is activation of the cytotoxic receptors on NK cells such as NKp44 and NKG2D that target cancer cells. For example, activation of NK cells by IL-2 induces expression of the natural cytotoxic receptor, NKp44, that is not expressed on resting NK cells (Vitale M et al, JEM, 1998, 187: 2065-2072) and NKp44 is the first activating NK cell receptor to recognise a tumour growth factor (Barrow Ad et al, Cell, 2018, 172(3): 534-548). Moreover, maximising NKG2D expression on immune cells remains a goal in cancer immunotherapy which may be achievable by use of IK14004 (Duan S et al, Molecular Cancer, 2019, doi.org/10.1186/s12943-019-0956-8; (Frederiksen K S et al, Cancer Immunol Immunother, 2008, 57 (10: 1439-49; Takaki R et al, J Immunol, 2005, 175(4): 2167-73). FIG. 18 demonstrates RSKAKNPLYR-(2Adod)₄-NH₂ (“IK14004”) enhances NKp44 expression on NK cells. FIG. 19 demonstrates RSKAKNPLYR-(2Adod)₄-NH₂ (“IK14004”) enhances NKG2D receptor expression on NK cells.

In one embodiment, the subject is a subject with cancer, and is selected for treatment to treat and/or prevent an autoimmune disorder during cancer therapy.

In one embodiment, the subject with cancer is undergoing cancer therapy and is selected for treatment to treat and/or prevent an autoimmune disorder during cancer therapy.

In another embodiment, the subject is receiving cancer therapy, and is selected for treatment with a peptide or a composition as described herein, to reduce immune related adverse events.

In a preferred embodiment, the subject is receiving checkpoint inhibitor therapy.

As used herein, homeostatic levels of IL-2 refers to levels of IL-2 that are effective for the treatment of autoimmune disease, but are not high enough to exacerbate autoimmune and/or inflammatory diseases (e.g. a therapeutic window of IL-2). It is known that high dose IL-2 can exacerbate a variety of autoimmune and inflammatory diseases.

In one embodiment, the present invention provides a method as described herein, wherein the effective amount of a peptide comprising an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂ modulates the activity of Lck and/or G-protein signalling to maintain levels of IL-2 in the subject that do not exacerbate an autoimmune and/or an inflammatory disease.

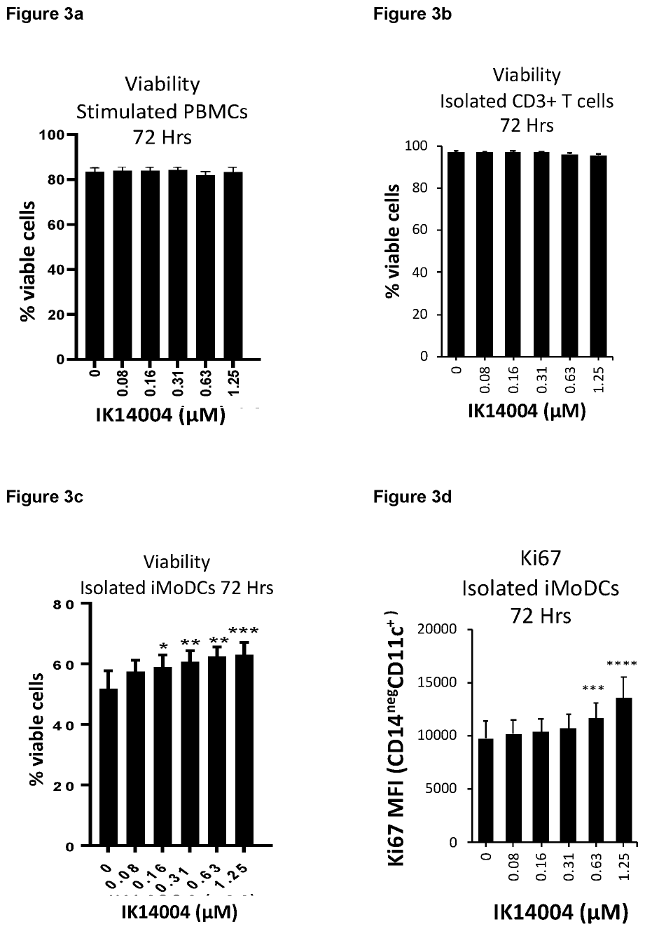

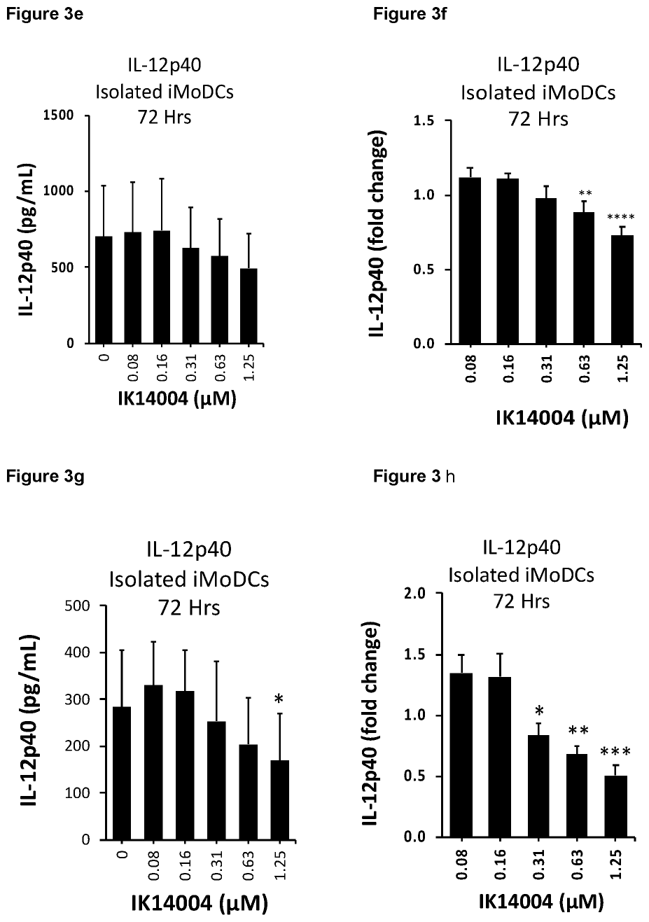

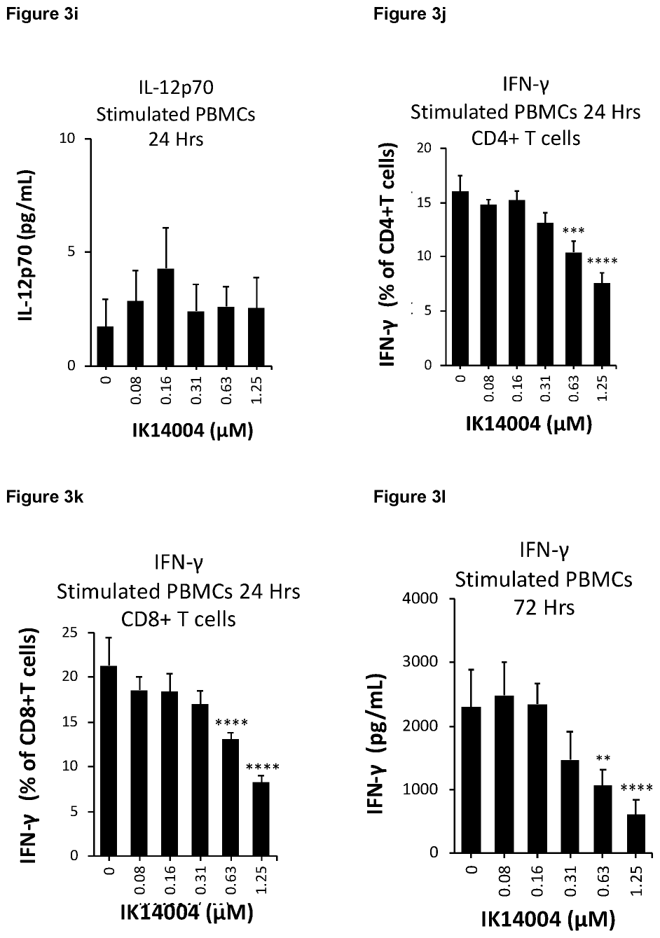

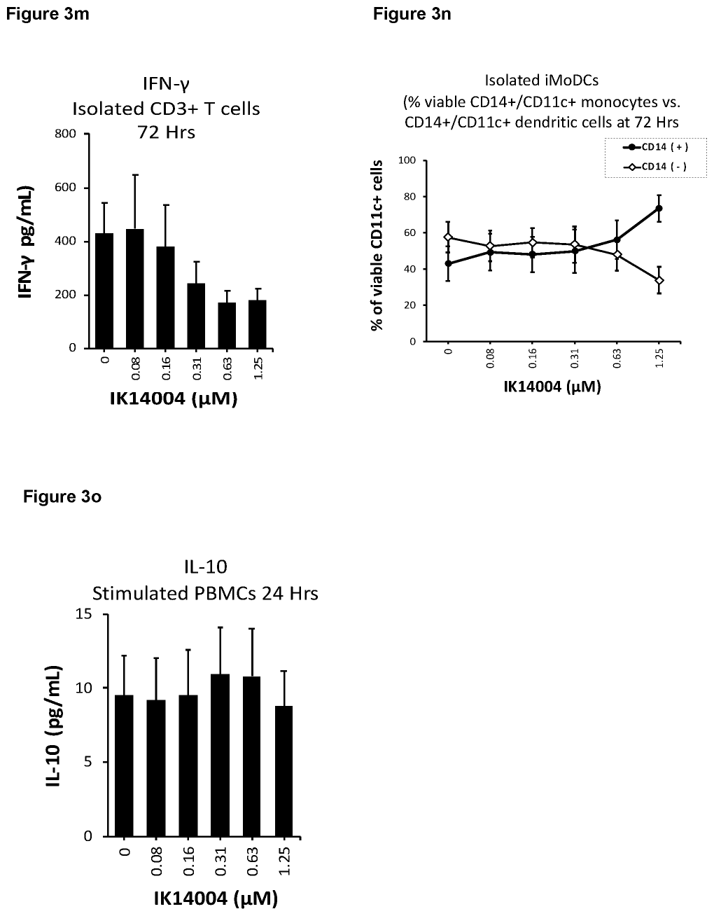

Activation of Lck is required for both positive and negative regulation of IL-2 production via different pools of Lck and relatively low levels of IL-2 produced by activated T cells in the presence of IK14004 combined with IK14004-mediated inhibition of inflammatory cytokines, ie, IL-12p40 and IFNg (FIG. 3 c-f and i-l), could serve to minimise autoimmune inflammatory responses.

The present inventors have demonstrated herein that Lck activation, for example by IK14004, unexpectedly does not lead to a commensurate increase in levels of IL-2. This suggests that there is a secondary effect of the peptides described herein that leads to high levels if IL-2 being avoided.

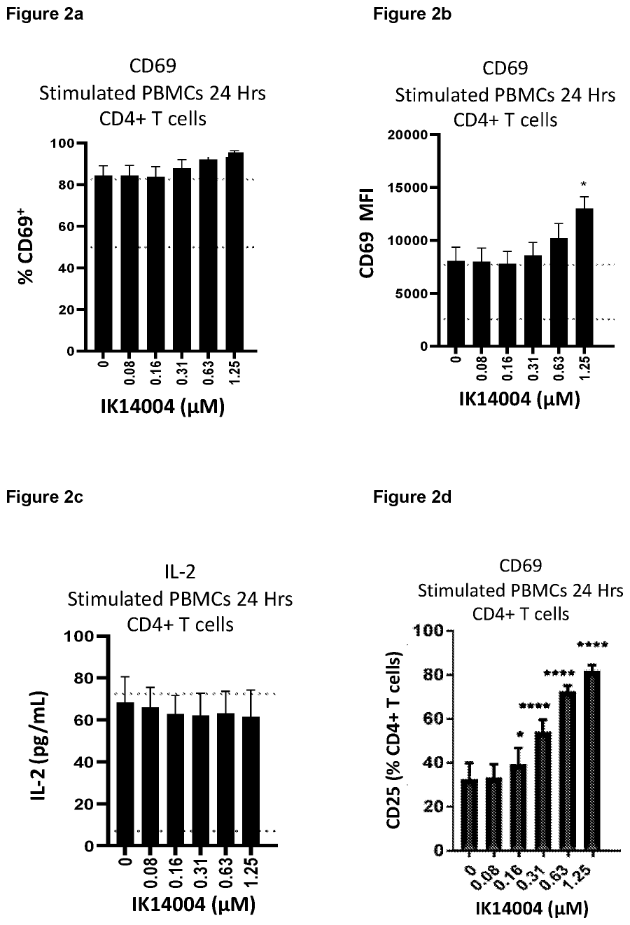

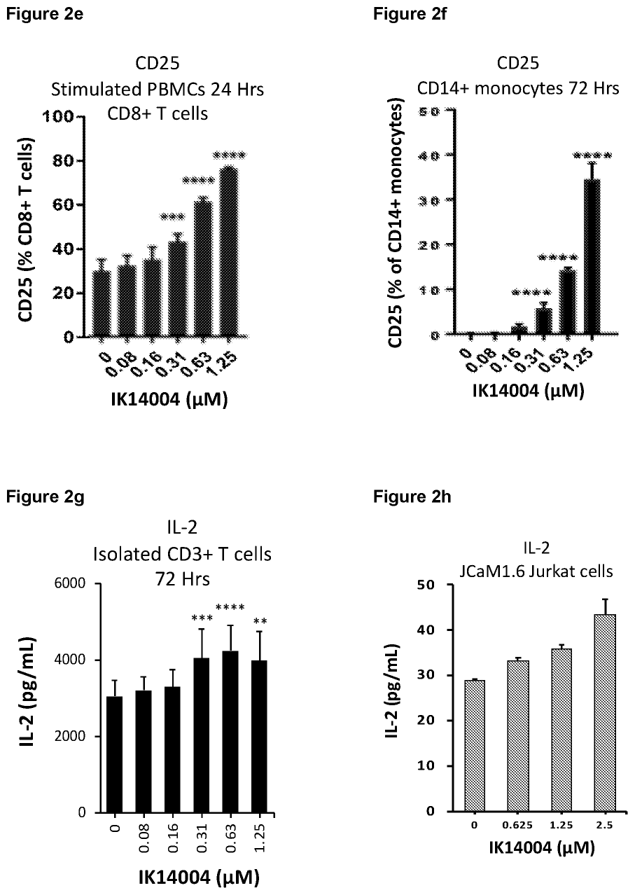

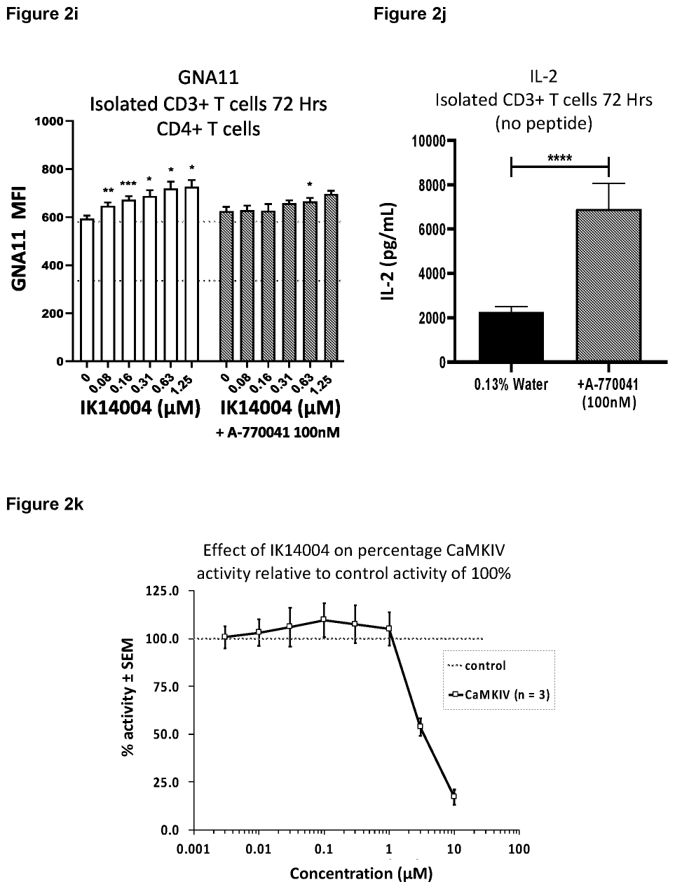

Taken together with the finding of IK14004-enhanced IL-2 production in Lck-deficient cells (FIG. 2 h) the present inventors propose, without wishing to be bound by theory, that IK14004-mediated signalling via GNA11 modulates G-protein-induced cytokine production. For example, stimulation of PBMCs and isolated monocytes by superantigens has been shown to result in increased production of IL-2, IL-12p40, and IFNg whereas IK14004 induces only a small increase in IL-2 from activated T cells whilst inhibiting production of IL-12p40 and IFNg.

Given the dominance of activated Lck within the internal pool of Lck compared with levels of active Lck at the plasma membrane, the present inventors propose that IK14004 contributes to maintenance of homeostasis by regulating immune responses in both Lck-dependent and -independent ways. Without wishing to be bound by theory, enhancement of Lck activity within the internal Lck pool would serve to retain phosphorylated NFAT1 within the cytosol and prevent IL-2 gene induction. Inhibition of CaMKIV activity would achieve the same purpose upon TCR activation and mobilisation of calcium stores.

Lck has been shown to contribute to down-regulation of T cell activation and cytokine production upon superantigen-induced T cell activation (Criado G & Madrenas J, J Immunol, 2004, 172(1): 222-230) and IK14004 may further enhance this down-regulation effect via a negative regulatory pathway that involves Gal 1. The inhibitory effect on CaMKIV activity (FIG. 2 k) combined with a ten-fold greater enhancement of IL-2 production observed in the presence of a Lck inhibitor, eg, A770041, (FIG. 2 i,j) compared with a Lck modulator, e.g, IK14004, suggests that IK14004-induced Lck activity may limit excessive production of IL-2 in resting and stimulated T cells.

As indicated above, the observed increase in Lck activity that was expected to result in a commensurate increase IL-2 levels did not lead to excessive production of IL-2. Instead, maintenance of a low-level increase in IL-2 levels was observed, which allows maintenance of IL-2 levels in a therapeutic window. Without wishing to be bound by theory, the present inventors propose that Lck signalling via G-proteins, or via TCRs, to result in IL-2 production, is tempered by the inhibitory effect of the peptides on CamKIV shown in FIG. 2.

Accordingly, the present inventors propose that IK14004 may act as a rheostat to maintain IL-2 homeostasis upon T cell activation.

In one embodiment the present invention provides a method as described herein, wherein the IL-2 producing cells are selected from the group consisting of B cells, T cells or dendritic cells.

The present inventors have demonstrated in Example 5 that RSKAKNPLYR-(2Adod)₄-NH₂ inhibits JAK/STAT signalling. Since the JAK family member Tyk2 plays a crucial role in mediating IL-23 receptor signalling and STAT3 activation and Tyk2 inhibitors are useful therapeutics for spondyloarthritis, the peptides described herein are suitable for use in treating spondyloarthritis.

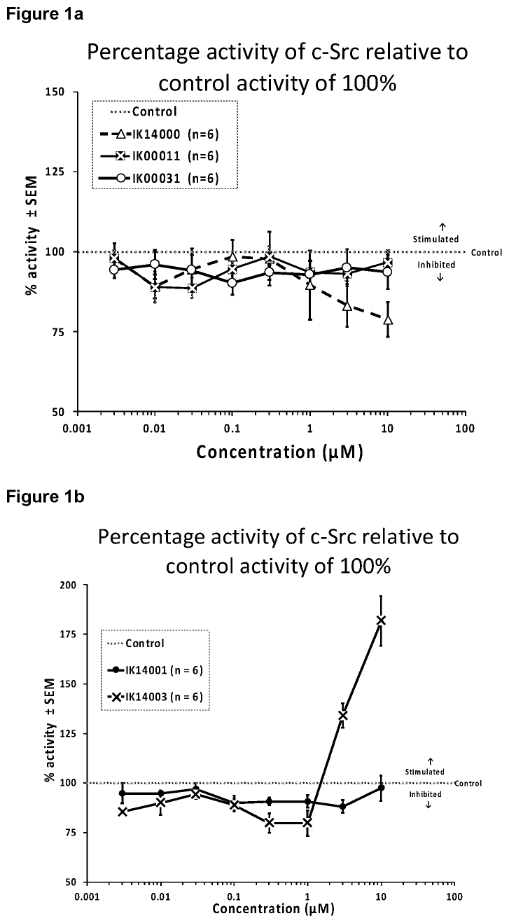

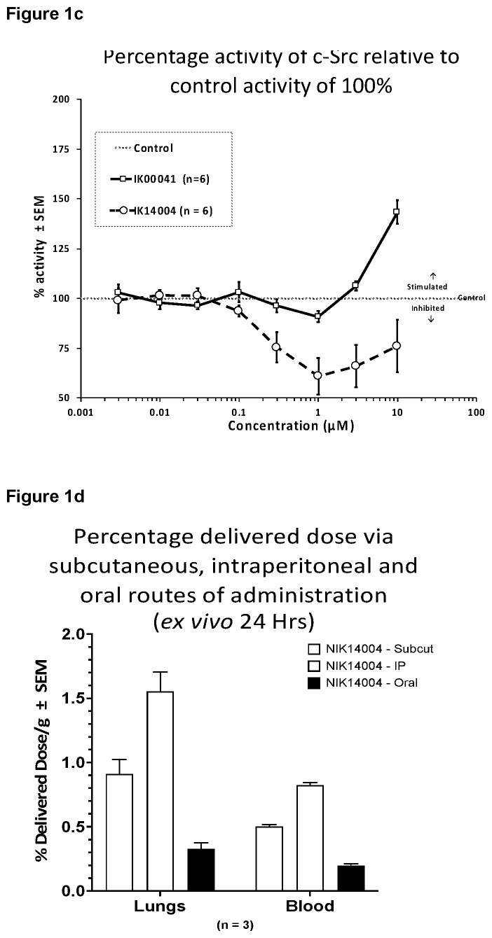

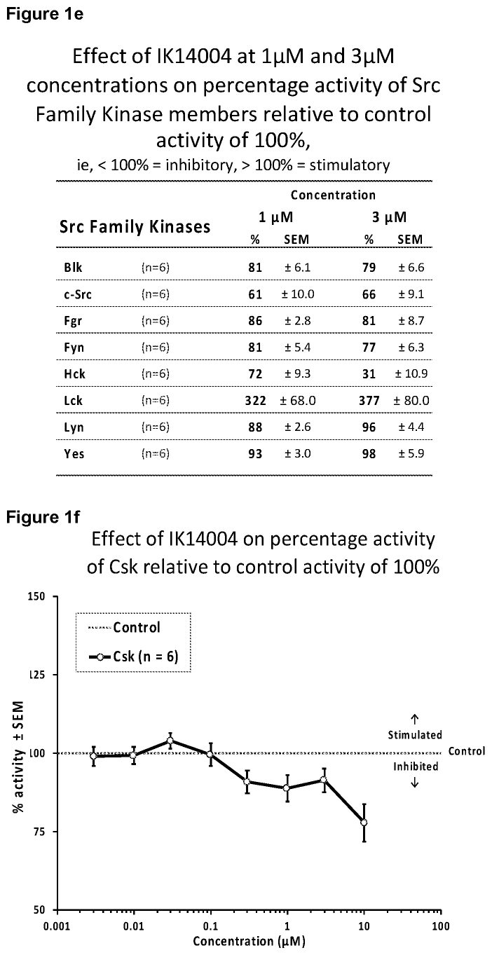

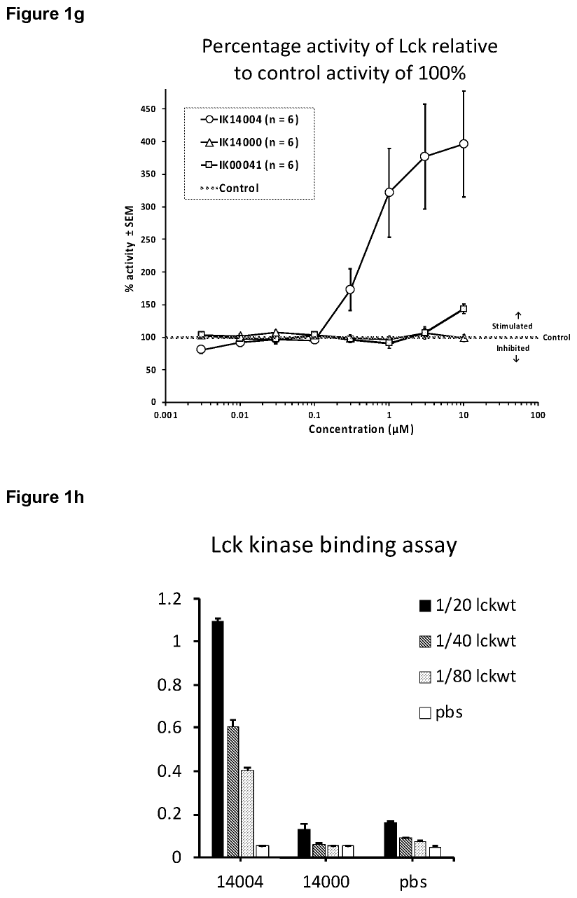

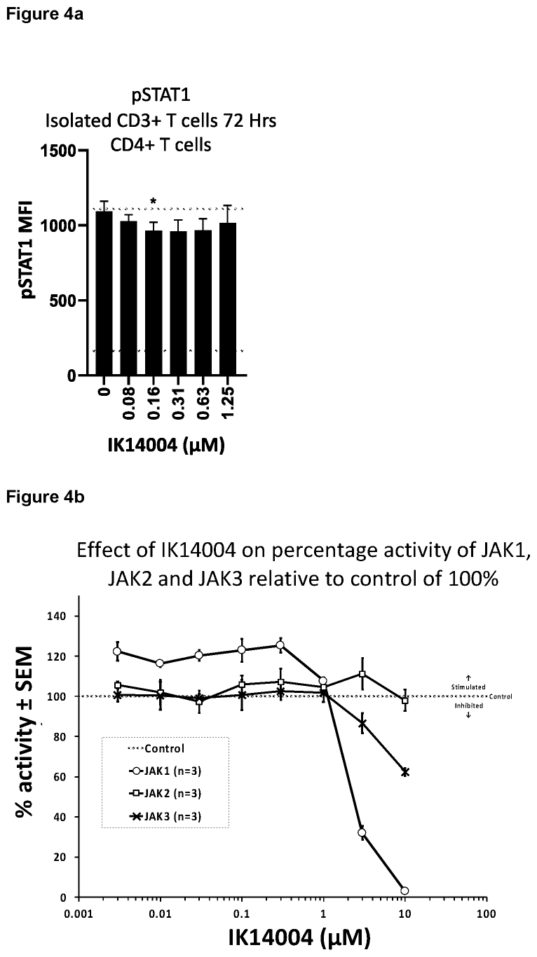

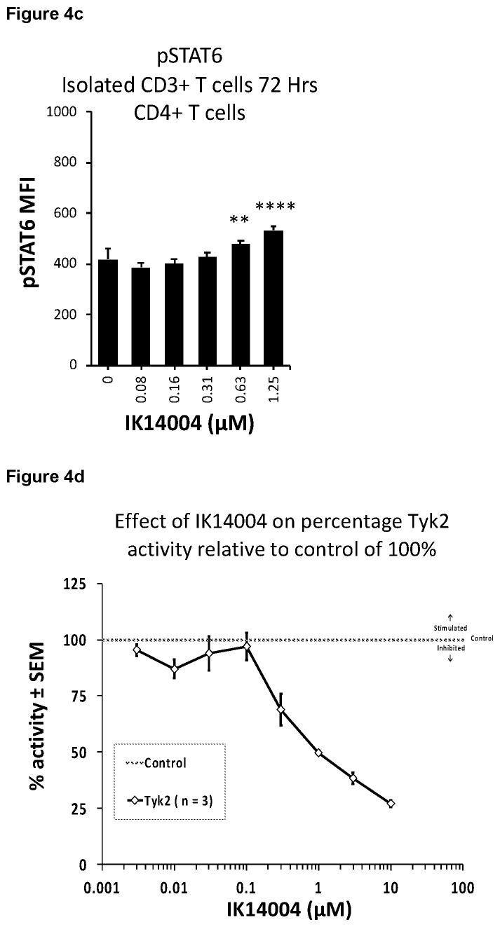

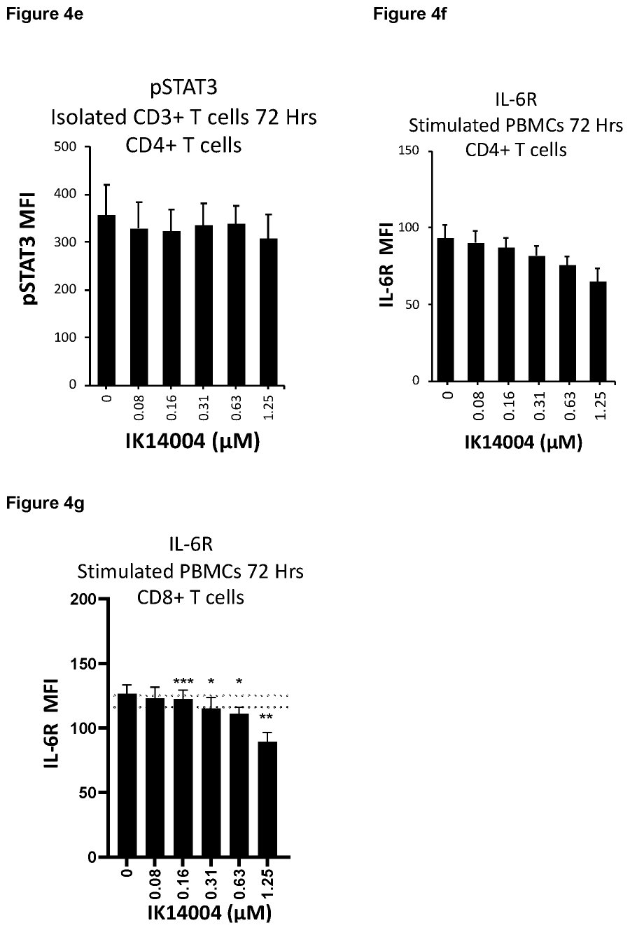

The treatment of the autoimmune manifestations of “gain-of-function” STAT1 and STAT3 disease has improved with use of Jakinibs, ie, JAK inhibitors (Forbes L R et al, J Allergy & Clinical Immunology, 2018, 142(5): P1665-P1669) for conditions such as rheumatoid arthritis, psoriasis and inflammatory bowel disease. Activation of STAT1 and STAT3 occurs downstream of IFNg and this activation plays a central role in inflammation enhanced by a positive feedback loop comprising the IL-6-STAT3 axis in which IL-6 is involved in terms of autoantibody as seen in SLE (Hirano vide supra; Ogata A & Tanaka T, Int J Rheumatol, 2012, doi.org/10.1155/2012/946048). However, Jakinibs are not without side-effects such as activation of latent tuberculosis (Maiga M et al, J Infect Dis, 2012, 205(11): 1705-1708), increased risk of viral infections such as Herpes zoster, and anaemia amongst others (Gilhar A et al, The Lancet, 2019, 393 (10169): P318-P319; Schwartz D M et al, Nat Rev Drug Discov, 2017, doi: 10.1038/nrd.2017.267). Consequently inhibition of c-Src, IFNg, c-Src, STAT1 (to a small extent), and JAK1, by IK14004 (FIG. 1 c,e, FIG. 3 j-m; FIG. 4 a,b) may offer an alternative given that IFNg enhances the association between c-Src and STAT1 (Chang Y-J et al, Mol Pharmacol, 2004, 65(3): 589-98; Hwang S-J, et al, Toxicology Letters, 2013, 220(2): 109-117) and that JAK1 is constitutively activated in Src-transformed cells (Campbell G S et al, JBC, 1997, 272(5): 2591-4). Moreover, the absence of an inhibitory effect on JAK2 activity could prove an advantage with respect to development of anaemia because hematopoietic growth factors signal through JAK2 (Schwartz et al, vide supra).

The present inventors have demonstrated in Example 5 that RSKAKNPLYR-(2Adod)₄-NH₂ inhibits MAP4K1 (HPK1) kinase activity.

Because MAP4K1 (HPK1) kinase activity is a negative regulator of the TCR-induced AP-1 response pathway that leads to IL-2 gene expression, the present inventors propose that inhibition of HPK-1 allows AP-1 transcription to proceed unhindered in the nucleus resulting in IL-2 being maintained in a therapeutic window, while NFAT1-mediated IFNg induction is suppressed.

For example, CD4+ T cell anergy prevents autoimmunity and generates regulatory T cell precursors (Kalekar L A et al, Nat Immunol, 2016, 17(3): 304-314). The transcription factor AP-1 plays a critical role in the transactivation of the IL-2 gene by binding to multiple regulatory elements in the IL-2 promoter (Liou J et al, Immunity, 2000, 12(4): 399-408). The activation of MAP kinases, eg, ERK, activates AP-1 and ERK activation is enhanced by CD28 engagement at the TCR and both Lck and CD28 are essential for this process (Carey K D et al, Molecular & Cellular Biology, 2020, doi.org/10.1128/MCB.20.22.8409-8419.2000). Hence, full activation of MAP kinases requires the presence of the TCR co-receptor CD28 (Tuosto L & Acuto O, Eur J Immuol, 1998, 28(7): 2131-42) and G-protein (G-alphaq/11) activation of ERK1/2 via either the protein kinase C-Raf signalling axis or the calcium-calmodulin pathway appears to be cell-type dependent (Goldsmith Z G & Dhanasekaran D N, Oncogene, 2007, 26: 3122-2142). Consistent with this, the present inventors have demonstrated in Example 5 that RSKAKNPLYR-(2Adod)₄-NH₂ increased CD28 levels on CD4+ T cells.

Moreover, calcineurin inhibitors activate the Ras-Raf-MAP kinase pathway (Datta D et al, Cancer Res, 2009, doi: 10.1158/0008-5472.CAN-09-1404). Notably, the terminal centre kinase HPK1 is a negative regulator of the TCR-induced AP-1 response pathway that leads to IL-2 gene induction (Liou et al, vide supra) and given that decreased AP-1 has been observed in psoriasis and SLE (Trop-Steinberg S & Azar Y, Am J Med Sci, 2017, 353(3): 474-483), inhibition of both HPK1 and calcineurin (via inhibition of CamKIV) by IK14004 combined with enhanced CD28 expression in CD4+ T cells at the TCR may serve to achieve IL-2-regulated homeostasis. Taken together, IK14004 may indirectly impact on NFAT1− and AP-1− induced gene induction leading to low amounts of IL-2 produced whilst inhibiting IFNg via modulation of signalling events at the TCR and/or G-protein receptors, especially, in the presence of superantigens or endotoxins.

Bacterial LPS plays a role in some diseases in which self-antigen-specific T cells are involved (Yoshino S et al, Immunology; 2000, 99(4): 607-614; Granholm NA & Cavallo T, Lupus, 1994, doi.org/10.1177/096120339400300614). LPS induces IL-12p40 but not IL-12p70 in monocytes (Isler P et al, Amer J Resp Cell & Mol Biol, 1998, doi.org/10.1165/ajrcmb.20.2.3313) and the stimulatory role of IL-12p40 in GVHD and psoriasis is well-recognised (Toichi E et al, J Immunol, 2006, 177: 4917-4926; Cooper A M & Khader S A, Tends Immunol, 2007, 28(1): 33-8; Wu Y et al, Biol Blood Marrow Transplant, 2015, 21(7): 1195-1204). Given the role of IFNg in development of SLE (Liu W et al, BioMed Research International, 2020, doi.org/10.1155/2020/7176515) taken together with reported observations that a dominant negative NFAT molecule attenuates LPS- and IFNg-activated endogenous IL-12p40 mRNA expression (Zhu C et al, JBC, 2003, 278(41): 39372-39382) the present inventors propose that IK14004-mediated inhibition of IL-12p40 in DCs is either secondary to inhibition of IFNg production and/or de-stabilisation of DCs (FIG. 3n) may play a role in decreased IL-12p40 secretion in the presence of peptide.

In one embodiment the present invention provides a method as described herein wherein the effective amount of a peptide comprising an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂ does not induce IFNg and/or IL-12p40.

In one embodiment the present invention provides a method as described herein wherein the therapeutically effective amount of a peptide comprising an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂ is administered orally and/or topically.

In one embodiment the present invention provides a method as described herein wherein the peptide consists of an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂ In one embodiment the present invention provides a use of therapeutically effective amount of a peptide comprising an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂ in a method of treating or preventing an autoimmune disorder in a subject.

In one embodiment the present invention provides a use of a peptide comprising an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂ in the manufacture of a medicament for treating an autoimmune disorder in a subject.

In one embodiment the present invention provides an oral dose form comprising an effective amount of a peptide comprising an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂ for the treatment of an autoimmune disorder in a subject.

In one embodiment the present invention provides a method as described herein wherein the peptide is administered orally or topically.

### EXAMPLES

### Example 1: RSKAKNPLYR-(2Adod)₄-NH₂ Inhibits c-Src and Activates Lck

### Materials and Methods

For FIG. 1a, b, c, e, f, g, FIG. 5:

Blk, Lyn is incubated with 50 mM Tris pH 7.5, 0.1 mM EGTA, 0.1 mM Na3VO4, 0.1% β-mercaptoethanol, 0.1 mg/mL poly(Glu, Tyr) 4:1, 10 mM MgAcetate and [γ33P-ATP] (specific activity approx. 500 cpm/pmol, concentration as required). The reaction is initiated by the addition of the MgATP mix. After incubation for 40 minutes at room temperature, the reaction is stopped by the addition of 3% phosphoric acid solution. 10 μL of the reaction is then spotted onto a Filtermat A and washed three times for 5 minutes in 75 mM phosphoric acid and once in methanol prior to drying and scintillation counting.

c-Src, Fyn, Hck is incubated with 8 mM MOPS pH 7.0, 0.2 mM EDTA, 250 μM KVEKIGEGTYGVVYK (Cdc2 peptide), 10 mM MgAcetate and [γ33P-ATP] (specific activity approx. 500 cpm/pmol, concentration as required). The reaction is initiated by the addition of the MgATP mix. After incubation for 40 minutes at room temperature, the reaction is stopped by the addition of 3% phosphoric acid solution. 10 μL of the reaction is then spotted onto a P30 filtermat and washed three times for 5 minutes in 75 mM phosphoric acid and once in methanol prior to drying and scintillation counting.

Fgr, Yes is incubated with 8 mM MOPS pH 7.0, 0.2 mM EDTA, 0.1 mg/mL poly(Glu, Tyr) 4:1, 10 mM MgAcetate and [γ33P-ATP] (specific activity approx. 500 cpm/pmol, concentration as required). The reaction is initiated by the addition of the MgATP mix. After incubation for 40 minutes at room temperature, the reaction is stopped by the addition of 3% phosphoric acid solution. 10 μL of the reaction is then spotted onto a Filtermat A and washed three times for 5 minutes in 75 mM phosphoric acid and once in methanol prior to drying and scintillation counting.

Lck is incubated with 50 mM Tris pH 7.5, 0.1 mM EGTA, 0.1 mM Na3VO4, 250 μM KVEKIGEGTYGWYK (Cdc2 peptide), 10 mM Magnesium acetate and [γ33P-ATP] (specific activity approx. 500 cpm/pmol, concentration as required). The reaction is initiated by the addition of the Mg/ATP mix. After incubation for 40 minutes at room temperature, the reaction is stopped by the addition of phosphoric acid to a concentration of 0.5%. 10 μL of the reaction is then spotted onto a P30 filtermat and washed four times for 4 minutes in 0.425% phosphoric acid and once in methanol prior to drying and scintillation counting.”

CaMKIV is incubated with 40 mM HEPES pH 7.4, 5 mM CaCl2, 30 μg/mL calmodulin, 30 μM KKLNRTLSVA, 10 mM MgAcetate and [γ-33P-ATP] (specific activity approx. 500 cpm/pmol, concentration as required). The reaction is initiated by the addition of the MgATP mix. After incubation for 40 minutes at room temperature, the reaction is stopped by the addition of 3% phosphoric acid solution. 10 μL of the reaction is then spotted onto a P30 filtermat and washed three times for 5 minutes in 75 mM phosphoric acid and once in methanol prior to drying and scintillation counting.

JAK1 is incubated with 20 mM Tris/HCl pH 7.5, 0.2 mM EDTA, 500 μM GEEPLYWSFPAKKK, 10 mM MgAcetate and [γ33P-ATP] (specific activity approx. 500 cpm/pmol, concentration as required). The reaction is initiated by the addition of the MgATP mix. After incubation for 40 minutes at room temperature, the reaction is stopped by the addition of 3% phosphoric acid solution. 10 μL of the reaction is then spotted onto a P30 filtermat and washed three times for 5 minutes in 75 mM phosphoric acid and once in methanol prior to drying and scintillation counting.

JAK2 is incubated with 8 mM MOPS pH 7.0, 0.2 mM EDTA, 100 μM KTFCGTPEYLAPEVRREPRILSEEEQEMFRDFDYIADWC, 10 mM MgAcetate and [γ33P-ATP] (specific activity approx. 500 cpm/pmol, concentration as required). The reaction is initiated by the addition of the MgATP mix. After incubation for 40 minutes at room temperature, the reaction is stopped by the addition of 3% phosphoric acid solution. 10 μL of the reaction is then spotted onto a P30 filtermat and washed three times for 5 minutes in 75 mM phosphoric acid and once in methanol prior to drying and scintillation counting.

JAK3 is incubated with 8 mM MOPS pH 7.0, 0.2 mM EDTA, 500 μM GGEEEEYFELVKKKK, 10 mM MgAcetate and [γ33P-ATP] (specific activity approx. 500 cpm/pmol, concentration as required). The reaction is initiated by the addition of the MgATP mix. After incubation for 40 minutes at room temperature, the reaction is stopped by the addition of 3% phosphoric acid solution. 10 μL of the reaction is then spotted onto a P30 filtermat and washed three times for 5 minutes in 75 mM phosphoric acid and once in methanol prior to drying and scintillation counting.

TYK2 is incubated with 8 mM MOPS pH 7.0, 0.2 mM EDTA, 250 μM GGMEDIYFEFMGGKKK, 10 mM MgAcetate and [γ33P-ATP] (specific activity approx. 500 cpm/pmol, concentration as required). The reaction is initiated by the addition of the MgATP mix. After incubation for 40 minutes at room temperature, the reaction is stopped by the addition of 3% phosphoric acid solution. 10 μL of the reaction is then spotted onto a P30 filtermat and washed three times for 5 minutes in 75 mM phosphoric acid and once in methanol prior to drying and scintillation counting.

For FIG. 1d, 1,4,7-Triazacyclononane-1,4,7-triacetic acid (NOTA) conjugated peptides were acquired from Auspep, reconstituted in deionised water at 10 mg/mL and used without further purification. Peptides were incubated with ⁶⁴Cu at 1000-fold excess of peptide in 0.1 M pH 5.5 ammonium acetate buffer for 45 minutes at 37° C. 1 μL Samples of each solution were taken and mixed 1:1 with 50 mM EDTA. EDTA incubated sample or neat solution was spotted on TLC paper (Agilent iTLC-SG Glass microfiber chromatography paper impregnated with silica gel) and run with 50:50 H2O:ethanol. Detection of radiolabelled species migration was then achieved by imaging on a Bruker In Vivo MS FX Pro imaging system using a radioisotopic phosphor screen. Control experiments were conducted to monitor the elution profile of free ⁶⁴Cu and ⁶⁴Cu bound to EDTA for quality control. All samples showed 100% radiolabelling purity. Labelled peptides were then diluted in deionised water prior to administration to achieve required specific activity.

Mice were anaesthetised using 2% isofluorane in 02 for all injection and imaging procedures throughout. Female C57 mice (approximately 8 weeks of age) were injected (29G needle, 50 μL Aqueous solution, 3.5 MBq [⁶⁴Cu]NOTA-peptide) intraperitoneal with radiolabelled peptides.

PET-CT imaging utilized a Siemens Inveon PET-CT scanner with physiological monitoring achieved using a respiratory probe (BioVet™ system, m2m Imaging, Australia). Dynamic PET-CT images were acquired for 45 minutes from 5 minutes post peptide administration. Static images were then acquired at 8 and 24 hours post administration (30 minutes each). Following each PET acquisitions, micro-CT scans were acquired for anatomical co-registration. The CT images of the mice were acquired through an X-ray source with the voltage set to 80 kV and the current set to 500 μA. The scans were performed using 360° rotation with 120 rotation steps with a low magnification and a binning factor of four. The exposure time was 240 ms with an effective pixel size of 106 μm. The total CT scanning process took approximately 15 minutes. The CT images were reconstructed using Feldkamp reconstruction software (Siemens).

The PET Images were reconstructed using an ordered-subset expectation maximization (OSEM2D) algorithm and analysed using the Inveon Research Workplace software (IRW 4.1) (Siemens) which allows fusion of CT and PET images and definition of regions of interest (ROIs). A dynamic tracer uptake profile was obtained by a reconstruction of the dataset into 5 minute frames. CT and PET datasets of each individual animal were aligned using IRW software (Siemens) to ensure good overlap of the organs of interest. Three dimensional ROIs were placed within the whole body, as well as all the organs of interest, such as heart, kidney, lungs, bladder, liver, spleen and tumour, using morphologic CT information to delineate organs. Activity per voxel was converted to nci/cc using a conversion factor obtained by scanning a cylindrical phantom filled with a known activity of ⁶⁴Cu to account for PET scanner efficiency. Activity concentrations were then expressed as percent of the decay-corrected injected activity per cm³ of tissue that can be approximate as percentage injected dose/g (% ID/g). For FIG. 1h, the plasmid for the expression of wild type Lck protein was kindly provided by the research lab of Professor Katharina Gaus, University of NSW. The plasmid encoded for mammalian cell expression of the Lck wild type protein with a C-terminal mCherry reporter. A twin-strep tag was incorporated at the N-terminal of the Lck-WT for purification, and a c-myc epitope tag was placed at the C-terminal, after mCherry for detection of protein expression. A midiprep DNA preparation of the plasmid was performed using the Macharey-Nagal midi kit as per manufacturer's instructions. For transient transfections plasmid DNA was transfected into CHO-S cells using 2 μg DNA mL-1 cells at a concentration of 3 million cells mL-1. DNA was complexed with polyethylenimine-Pro (PolyPlus) in Opti-Pro serum free medium (Life Technologies) at a DNA (μg) to PEI (μL) ratio of 1:4 (w:v) for 15 min prior to transfecting suspension adapted CHO cells. The transfected cells were cultured in chemically defined CHO medium (CD-CHO; Life Technologies) at 37° C., 7.5% C02, 70% humidity with shaking at 130 rpm for 6 hrs, before feeding with 7.5% CD CHO Efficient Feed A (Life Technologies), 7.5% CD-CHO Efficient Feed B (Life Technologies), and 0.4% anti-clumping agent (Gibco) and continuing the culture at 32° C., 7.5% CO₂, 70% humidity with shaking at 130 rpm for 2 days. Following transfection, the cells were pelleted by centrifugation at 5250 g for 30 min. The cells were briefly sonicated with 2 pulses of 30 secs on/off using the Vibra Cell VC505 sonicator (Sonics). Cells were centrifuged at 5250 g for 10 mins and supernatant was collected and filtered through a 0.22 μm membrane (Sartorius). The Lck-WT protein was purified from the clarified supernatant using a 5 mL Strep-Trap column (GE). Protein was eluted using desthiobiotin.

Lck-Peptide binding ELISAs. The peptide IK14004 was reconstituted into PBS at 1 mg/mL. Each peptide stock was diluted in PBS to 10 μg/mL and 100 μL added to wells of a maxisorp plate (Nunc). Wells were coated at 40° C. for 20 hrs. Triplicate wells were used for testing the binding of different concentrations of the purified Lck-WT protein. Following coating, the wells were blocked with 200 μL of 2% Milk-PBST (0.05% Tween 20 in PBS) for 1 hr. The blocker was then decanted and 100 μL of Lck-WT diluted in PBS was added and incubated at room temperature for 2 hrs. The wells were then washed 4 times with PBST (200 μL per well/wash) and 100 μL of HRP anti-myc (Miltenyi Biotech) diluted 1/5000 in block solution was added for 1 hr at room temperature. The wells were then washed 4×PBST and 100 uL of TMB (Sigma) was added per well for 10 mins. 100 μL of 2M sulphuric acid was added to stop the reaction and the absorbance values recorded at a wavelength of 450 nm using the Spectramax (Molecular Devices).

The present inventors sought to compare the effect of a single lipidic peptide residue based on saturated dodecanoic acid, ie, hyperbranched at the carbon 2 position (2Adod1)(designated IK00011) and a lipidic peptide comprising 3 residues (2Adod3)(designated IK00031) linked together in a linear sequence with the known effect of RSKAKNPLYR (designated IK14000) on inhibition of c-Src activity (Agrez et al). Contrary to expectations, neither the single residue (IK00011) nor three linked residues (IK00031) enhanced c-Src activity compared with RSKAKNPLYR (designated IK14000) (FIG. 1a), which inhibits c-Src.

Accordingly, the 10 mer RSKAKNPLYR was conjugated to IK00011 and IK00031, respectively, and RSKAKNPLYR-(2Adod3)(designated IK14003) stimulated c-Src activity above 1 uM whereas RSKAKNPLYR-(2Adod1) (designated IK14001) did not (FIG. 1b). To confirm this c-Src-activation trend observed with increased number of lipidic peptides, 4 residues were conjugated to the 10 mer, ie, RSKAKNPLYR-(2Adod4) (designated IK14004), and further residues could not be added because of insolubility. In contrast to c-Src activation in the presence of RSKAKNPLYR-(2Adod3) (designated IK14003) at concentrations above 1 uM, IK14004 effectively inhibited c-Src activity at these concentrations compared with the unconjugated lipidic peptide comprising 4 residues (IK00041) which induced c-Src activity as also seen for IK14003 (FIG. 1b).

To determine whether these inhibitory in vitro concentrations of IK14004 were achievable in vivo, uptake of Cu64-labelled NOTA-IK14004 in the lung was compared with blood levels after various routes of administration following ethics approval and according to methods detailed in the Supplementary Notes. Well tolerated doses of IK14004 given subcutaneously (SC), intraperitoneally (IP) and by oral gavage (OG), ie, 200 ug SC/IP and 1 mg OG, respectively, indicated that the low c-Src inhibitory concentrations for IK14004 in kinase profiling assays (0.3 uM-3 M) are achievable at commensurate tissue levels in vivo at 24 hours following a single dose of IK14004 (FIG. 1d) (as depicted by the grey shaded zone in the following kinase activity graphs). We then determined the effect of IK14004 on kinase activity for SFK members and, surprisingly, IK14004 induced activation of only Lck (FIG. 1e—Table). Lck activity is tightly controlled by conformational changes mainly relying on phosphorylation and dephosphorylation on the two regulatory tyrosine kinases, Tyr394, and the inhibitory residue Tyr505, and when c-terminal Src kinase (Csk) phosphorylates Tyr505 residue it results in an intra-molecular arrangement that locks Lck in an inactive or “closed” conformation (Rossy J et al, Front Immunol, 2012, 10.3389/fimmu.2012.00167). In contrast to Lck activation, IK14004 induced a slight dose-dependent inhibitory effect on Csk activity (FIG. 1f) suggesting the IK14004 acts to maintain Lck in the active state. Moreover, the conjugated peptide, IK14004, synergistically enhanced Lck activity since neither the 10 mer RSKAKNPLYR (designated IK14000) nor 4 lipidic peptide residues designated IK00041 activated Lck (FIG. 1g). While the mechanism of IK14004-mediated Lck activation was not determined, a direct activating effect of IK14004 on Lck was implied by binding assays that showed concentration-dependent binding of IK14004 to Lck in contrast to RSKAKNPLYR (IK14000)(FIG. 1h).

These data indicate that RSKAKNPLYR-(2Adod)₄-NH₂ inhibits c-Src and activates Lck.

FIG. 5 demonstrates that RSKAKNPLYR-(2Adod)₂-NH₂, like RSKAKNPLYR-(2Adod)₄-NH₂, inhibits c-Src and activates Lck. The present inventors have also demonstrated (data not shown) that RSKAKNPLYR-(2Adod)₂-NH₂ behaves similarly to RSKAKNPLYR-(2Adod)₄-NH₂ at 1 and 3 uM.

### Example 2: RSKAKNPLYR-(2Adod)₄-NH₂ Induces Expression of CD69 and CD25, and Lck-Independent IL-2 Production

The present inventors sought to determine whether IK14004 induced expression of activation markers on T cells following anti-CD3 antibody activation of peripheral blood mononuclear cells ((PBMCs) isolated from buffy coats obtained from healthy volunteers.

In brief, PBMCs were cultured for 24 hrs together with vehicle control (0.13% H2O) or IK14004 (0.08-1.25 μM), and stimulated with soluble anti-CD3 (1 μg/mL) or left unstimulated. After 24 hrs, cells were recovered and stained for CD69 and expression assessed by flow cytometry.

IK14004 induced a dose-dependent increase in expression of CD69 and CD25 as assessed by flow cytometry (FIG. 2 a,b,d,e).

To determine whether IK14004 induces IL-2 production, supernatants obtained from stimulated anti-CD3 stimulated PBMC cultures after 24 and 72 hours were assessed by ELISA.

In brief, PBMCs were cultured for 24 and 72 hrs together with vehicle control (0.13% H2O) or IK14004 (0.08-1.25 μM), and stimulated with soluble anti-CD3 (1 μg/mL) or left unstimulated. After 24 and 72 hrs, supernatants were collected and assessed for IL-2 concentration (pg/mL and fold change) by ELISA. Fold change in IL-2 production was determined by normalisation to the stimulated media only control.

No IL-2 was detected in the absence of peptide after 72 hours (data not shown) and after 24 hours detectable IL-2 secretion was not enhanced in the presence of IK14004 (FIG. 2c).

Given that this may have reflected binding and uptake of IL-2 by the high-affinity IL-2 receptor alpha chain (CD25) expressed on various cell subsets within a mixed PBMC population, CD25 expression on CD4+/CD8+ T cells and monocytes was examined.

In brief, freshly isolated PBMCs were stimulated with or without (−aCD3) anti-CD3 (1 μg/mL) for 24 hrs in the presence of test peptide IK14004 at indicated concentrations (pM). CD14+ monocytes were isolated from fresh PBMCs and cultured for 72 hrs in the presence of test peptides over a 5-point concentration curve plus vehicle (0-1.25 μM) and anti-CD3 (1 μg/mL). After 72 hrs, cells were assessed for CD25, Ki67, IL-12Rβ1 and IL-12Rβ2 expression by flow cytometry.

CD25 expression was confirmed for CD4+/CD8+ T cells and monocytes (FIG. 2d,e,f) in PBMC cultures.

To examine IL-2 secretion, Isolated T cells (CD3+) were stimulated with anti-CD3 anti-CD28 Dynabeads™ and cultured together with the peptide IK14004 over a 5-concentration range plus vehicle control (0-1.25 μM) for 72 hrs after which supernatants were collected and assessed for IL-2 by ELISA. In contrast to absence of IL-2 in supernatants from PBMC cultures after 72 hours, IK14004 significantly enhanced IL-2 production by isolated, anti-CD3/anti-CD28-stimulated CD3+ T-cells (FIG. 2g).

The conventional paradigm of T cell activation through the TCR states that Lck plays a critical role in this signalling process (Criado G & Madrenas J, J Immunol, 2004, 172(1): 222-230). Historically this has been confirmed in a somatically mutated leukemic T-cell line that has a defect in the expression of functional Lck tyrosine kinase, ie, Jurkat CaM1.6 cells, and expression of Lck cDNA in JCaM1.6 restores the ability of the cell line to respond to TCR stimulation indicating that Lck is required for normal signal transduction through the TCR (Straus DB & Weiss A, Cell, 1992, 70(4): 585-93). J.CaM1.6 cells activated with plate-bound anti-CD3 and soluble anti-CD28 were used to confirm that exposure to IK14004 would not enhance IL-2 production.

In brief, a 12 well-plate was coated with a solution of anti-CD3 (5 μg/ml) made up in PBS (total volume 250 μl/well) and incubated overnight at 37° C. The coating solution was aspirated off and the coated wells gently washed with PBS (twice, 1 ml, 5 minutes) JCaM1.6 cells were seeded at (1×106 cells/well) in the “anti-CD3 coated wells” and subsequently stimulated with anti-CD28 (5 μg/ml), as well as treated with various concentrations of peptide IK14004 (0, 0.625, 1.25 and 2.5 μM) The cells were then incubated for 48 hours at 37° C. Cell suspensions were checked under the microscope, then transferred into 2 ml labelled tubes and centrifuged at 30,000 g for 10 minutes. The supernatant and pellet were separated for each sample and the supernatant (100 μl, n=3) analysed for the IL-2 content using ELISA.

Unexpectedly, IK14004 enhanced IL-2 production in a dose-dependent manner (FIG. 2h) indicating that the peptide could also stimulate a Lck-independent signalling pathway.

### Example 3: RSKAKNPLYR-(2Adod)₄-NH₂ Enhances Expression of GNA11 on Normal Human T-Cells, and Maintains IL-2 Levels within a Therapeutic Window

G proteins are expressed ubiquitously on immune cells and the Lck-deficient Jurkat cell line is known to express the G protein receptor GNA11 responsible for G protein-mediated IL-2 production in the presence of superantigens (Bueno C et al, Immunity, 2006, 25: 67-78).

The present inventors sought to determine whether IK14004 enhances expression of GNA11 on normal human T-cells.

In brief, CD3+ T cells were cultured for 72 hrs together with vehicle control (0.13% H2O) or IK14004 (0.08-1.25 μM), or IK14004 (0.08-1.25 μM) in the presence of inhibitor A-770041 (100 nM) and stimulated with soluble anti-CD3 anti-CD28 stimulation beads (4:1 cell to bead ratio) or left unstimulated. After 72 hrs, cells were recovered and stained for GNA11, and expression assessed by flow cytometry. GNA11 expression was detected using a PE conjugated donkey F(ab′2) anti-rabbit IgG H&L antibody.

A dose-dependent increase in GNA11 expression was observed on CD4+ T cells within stimulated, isolated CD3+ T cell cultures after 72 hours (FIG. 2i). This raised the possibility of IK14004-mediated Lck-independent effects on an internal pool of active Lck known to exert a negative regulatory pathway of T cell activation as seen in the presence of superantigens and Lck inhibitors (Criado G & Madrenas J, J Immunol, 2004, 172(1): 222-230; Baer A et al, PLOS One, 2017, doi.org/10.1371/journal.pone.0187123). Importantly, the internal Lck pool contains more activated Lck than at the TCR (Wei Q et al, PNAS, 2020, 117(27):15809-15817).

Given the opposing roles of the different pools of activated Lck within resting T cells and TCR-activated cells (internal negative regulatory Lck pool versus the positive regulatory pool of membrane-bound Lck), the present inventors examined the effect of a Lck inhibitor, A-770041, on IL-2 production in isolated CD3+ T-cell cultures.

In brief, CD3+ T cells were cultured for 72 hrs together with vehicle control (0.1% DMSO), the small molecule inhibitor A-770041 (100 nM), and stimulated with soluble anti-CD3 anti-CD28 stimulation beads (4:1 cell to bead ratio) for 72 hrs. After 72 hrs, supernatants were collected and assessed for IL-2 concentration (pg/mL) by ELISA.

Compared with a 30% increase in IL-2 secretion induced by IK14004 (FIG. 2g), the Lck inhibitor induced a 300% increase above vehicle control values (FIG. 2j). Notably, activated Lck appeared to contribute to G protein expression given that IK14004-mediated enhancement of GNA11 expression was less pronounced in the presence of A-770041 (FIG. 2i).

The internal negative regulatory pool of active Lck indirectly controls a variety of inducible genes such as IL-2, IFNg and TNF alpha via transcription factors of the nuclear factor of activated T cells, NFAT, (Kiani A et al, Blood, 2000, 98(5): 1480-8; Teixeira L K et al, J Immunol, 2005, 175(9): 5931-9). Activated Lck phosphorylates NFAT1 which serves to retain NFAT1 in the cytosol and thus prevent production of IL-2 and IFNg (Baer A et al, vide supra). On the other hand, TCR-mediated signalling results in activation of calcineurin via calcium-calmodulin kinase (CaMKIV) (Liu J O, Immunol Rev, 2009, 228(1): 184-198) which dephosphorylates NFAT1 resulting in its translocation into the nucleus. Hence, the present inventors sought to determine whether IK14004 affects CaMKIV activity. Consistent with the regulatory role for IK14004 in preventing excessive Th1 cytokine expression upon TCR-pMHC engagement by maintaining Lck-mediated phosphorylation of NFAT1, the peptide also inhibits the activity of CaMKIV as assessed in non-cell-based kinase profiling (FIG. 2k).

These results suggest that Lck signalling—via G-proteins or via TCRs—to result in IL-2 production, is tempered by the inhibitory effect of the peptides on CamKIV, resulting in a low-level increase of IL-2, and avoiding high levels of IL-2.

### Example 4: RSKAKNPLYR-(2Adod)₄-NH₂ Inhibits IL-12p40 and IFNg

The present inventors confirmed IK14004 does not affect the viability of cultured PBMCs, isolated CD3+ T cells, and isolated immature monocyte-derived dendritic cells (DCs)(FIG. 3a,b,c) including enhanced proliferation of isolated DCs (FIG. 3d).

The roles of IL-12 and IFNg cytokines as therapeutic targets in autoimmune diseases has recently become more defined (Sun L et al, Cytokine, 2015, 75(2): 249-55; Liu W et al, BioMed Research international, 2020, Article ID 7176515; Schurich A et al, Rheumatology, 2018, 57(2): 246-254; Adorini L et al, Cellular & Mol Life Sciences CMLS, 1999, 55: 1610-1625) and it is now proposed that IL-12p40 instead of IL-12p70 is the major driving force behind pathology associated with autoimmune diseases (Khader SA & Thirunavukkarasu S, J Immunol, 2019, 202(3): 629-630). Hence, the features attributed to IL-12 and T helper cell differentiation with respect to autoimmune diseases appear to be dependent on the p40 subunit (Kreymborg K et al, Exp Opin Ther Targets, 2005, 9(6): 1123-1136). Accordingly, the present inventors sought to compare the effect of IK14004 on IL-12 production for isolated monocyte and anti-CD-stimulated PBMC cultures and whereas IL-12p40 was inhibited (FIG. 3e,f,g,h), the production of IL-12p70 was unaffected (FIG. 3i).

IL-12 acts as a potent inducer of IFNg production by T-cells (Ma X et al, J Exp Med, 1996, 183: 147-157; Tugues S et al, Cell Death & Differentiation, 2015, 22: 237-246) and IFNg acts in a positive feedback loop to induce IL-12 at a transcriptional level (Ma et al, vide supra; Bhat P et al, Cell Death Dis, 2017, 8(6): e2836).

Hence, the present inventors wished to assess the effect of IK14004 on IFNg production and, similar to IL-12p40, dose-dependent inhibition of IFNg production was observed from stimulated PBMC and isolated CD3+ T cell cultures (FIG. 3j,k,l,m).

In contrast to IL-12, the major role of IL-10 is to maintain a homeostatic state (Ma X et al, F1000Res, 2015, doi: 10.12688/f1000research.7010.1). IL-10 is produced primarily by pathogen-activated antigen-presenting cells and plays a crucial role in preventing inflammatory and autoimmune pathologies (Iyer S S & Cheng G, Crit Rev Immunol, 2013, 32(1): 23-63). In addition, the potential for DCs to induce either tolerogenic or inflammatory responses is well-recognised (Li H & Shi B, cellular & Molecular Immunology, 2014, 12: 24-30). Tolerogenic, less mature DCs are characterised by lower surface levels of MHC class II and costimulatory molecules (Wakkach A et al, Immunity, 2003, 18(5): 605-617; Li H & Shi B, Cellular & Molecular Immunology, 2014, 12: 24030).

To determine whether either of these anti-inflammatory pathways might be involved, the present inventors examined the effect of IK14004 on DC phenotype (CD14+ expression) and IL-10 production. Interestingly, the viable monocyte population increased (CD14+/CD11+) at the expense of the viable DC population (CD14−/CD11c+) (FIG. 3n). Moreover, IL-10 production was unaffected in the presence of peptide (FIG. 3o) suggesting that suppression of the DC population may be more relevant with respect to inhibition of IL-12 production.

### Example 5: RSKAKNPLYR-(2Adod)₄-NH₂ Inhibits JAK/STAT Signalling

In light of the effect of IK14004 on IFNg production, the present inventors sought to assess the effect of peptide on the JAK/STAT pathway given that IFNg-triggered phosphorylation of STAT1 promotes the interactions of JAK1-JAK2 and JAK1/2-STAT1 leading to phosphorylation of STAT1 by JAKs (Igarashi K et al, JBC, 1994, 269(20): P14333-P14336; Horvath C M, Sci STKE, 2004, doi: 10.1126/stke.2602004tr8; Wei J et al, J Immunol, 2015, doi: 10.4049/jimmunol.1501111).

To confirm that IK14004-mediated IFNy suppression was not associated with off-target effects in the JAK/STAT pathway, STAT1 phosphorylation and kinase activity of JAKs was assessed in isolated CD3+ T cell cultures and kinase profiling assays, respectively. IK14004 induced slight inhibition of pSTAT1 (FIG. 4a) and of the three JAK kinases, maximal inhibition of activity was observed for JAK1 (FIG. 4b). To further explore possible off-target effects on STAT activation, the effect of the peptide on STAT6 phosphorylation given that loss of STAT6 promotes autoimmune diseases (Lau M et al, J Autoimmunity, 2012, 39(4): 388-97) was examined. In contrast to STAT1, IK14004 enhanced STAT6 phosphorylation in CD4+ T cells in a dose-dependent manner (FIG. 4c).

Amongst the JAK family members Tyk2 plays a crucial role in mediating IL-23 receptor signalling and STAT3 activation and Tyk2 inhibitors have been proposed as the next blockbuster therapeutics for spondyloarthritis (Hromadova D et al, Front Genet. 2021, doi.org: 10.3389/fgene.2021.685280). Tyk2 suppression has relevance given that its biological activity appears to be mediated via the via the p40 subunit of IL-23 (Hamza T et al, Int J Mol Sci, 2010, 11(3): 789-806). The effect of IK14004 on Tyk2 kinase activity and STAT3 activation in non-cell-based assays and isolated CD3+ T cell cultures, respectively, was examined. Exposure of Tyk2 to IK14004 revealed an IC50 of 1 uM (FIG. 4d) and induced a slight inhibitory trend, albeit not-statistically significant, on STAT3 phosphorylation (FIG. 4e). Moreover, interleukin-6 (IL-6)-mediated CD4+ T cell STAT3 activation is also associated with disease activity in rheumatoid arthritis (Anderson A E et al, Ann Rheum, 2016, 75(2): 466-73). Accordingly, we sought to examine the effect of the peptide on IL-6 receptor expression and consistent with lack of effect on STAT3 activation, IK14004 inhibited IL-6 receptor expression in CD4+/CD8+ T cells (FIG. 4f,g).

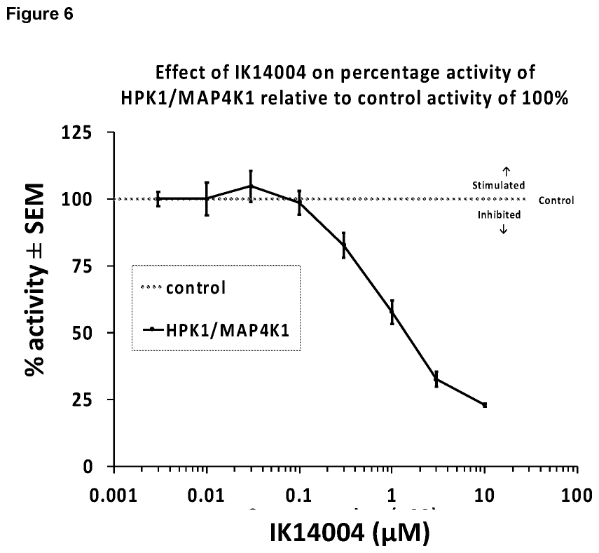

FIG. 6 demonstrates that RSKAKNPLYR-(2Adod)₄-NH₂ inhibits MAP4K1 (HPK1) kinase activity. HPK1, a human hematopoietic progenitor kinase activates the JNK/SAPK kinase cascade.

Because MAP4K1 (HPK1) kinase activity is a negative regulator of the TCR-induced AP-1 response pathway that leads to IL-2 gene expression, the present inventors propose that inhibition of HPK-1 allows AP-1 transcription to proceed unhindered in the nucleus resulting in IL-2 being maintained in a therapeutic window, while NFAT1-mediated IFNg induction is suppressed.

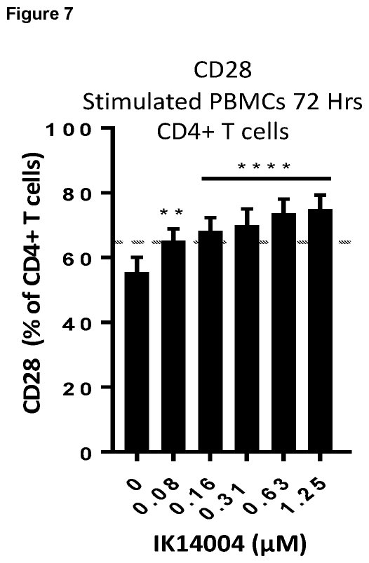

Because full activation of MAP kinases requires the presence of the TCR co-receptor, the present inventors sought to examine the expression of CD28 on T cells. In brief, PBMCs were cultured together with anti-CD3 (1 μg/ml) stimulation and IK14004 over a 5-concentration range (0-1.25 μM) for 72 hrs after which cells were assessed for expression of CD28 by flow cytometry. FIG. 7 demonstrates that RSKAKNPLYR-(2Adod)₄-NH₂ increased CD28 levels on CD4+ T cells.

T examine the ability of peptides comprising D-amino acids induce low levels of IL-2, IL-2 expression on exhausted CD4+ cells upon restimulation was examined. In brief, a single spleen per experiment was removed from MBP-Tracker mice and processed to generate a single cell suspension of splenocytes. Cells were resuspended at 3×10⁶/mL and stimulated with WT-MBP (control, non-exhausted cells) or APL-MBP (to generate exhausted cells). Cells were stimulated for 72 hr. Following stimulation, T cells were purified by ficoll density gradient and subsequently re-plated at 2×10⁶/mL in 20U/mL IL-2 for four days. After 4 days, cells were collected, resuspended (4×10⁵/mL, final 2×10⁴ per well) and restimulated using irradiated APC (from B10PL×C57BL/6 mice, 4×10⁶/mL, final 2×10⁵ per well) a single dose of APL-MBP peptide, together with test or reference substance or appropriate controls for 72 hrs. Following culture, supernatants were collected and stored frozen (−20° C.) for subsequent assessments of IL-2 cytokine production supernatant by multiplexed immunoassays using a ProcartaPlex custom mouse 4 Plex assay (Lot number 141554000, expiry 30 Jun. 2017) ran according to manufacturer instructions.

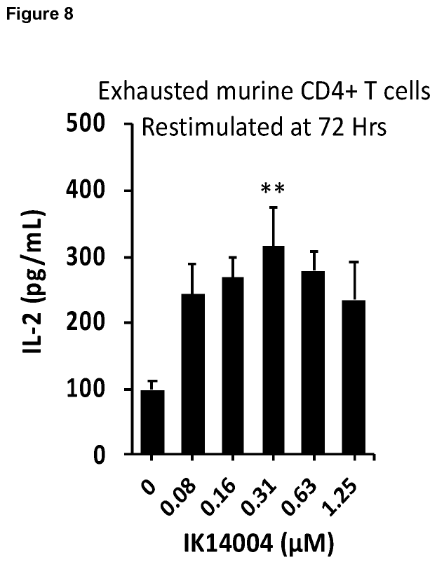

FIG. 8 demonstrates that rskaknplyr-(2Adod)₄-NH₂ (RSKAKNPLYR-(2Adod)₄-NH₂ comprising D amino acids) increases IL-2 expression on exhausted CD4+ cells upon restimulation.

### Example 6: RSKAKNPLYR-(2Adod)₄-NH₂ Increases the Proportion of CD25-Expressing Cells that Also Expressed Foxp3

IFN-γ is known to drive Treg fragility.

In light of the effect of IK14004 on IFNg production, the present inventors sought to assess the effect of IK14004 on Tregs within stimulated PBMC cultures after 72 hours.

Staining for T regulatory (Treg) cells was performed using anti-Foxp3 (PE conjugate; BioLegend) within CD4+/CD127^{low}/CD25+ T cells following fixation and permeabilization (Foxp3 transcription factor fixation buffer; ThermoFisher).

The proportion of CD25-expressing cells that also expressed Foxp3 increased in the presence of IK14004 (FIG. 9) which was reflected in the CD4/Treg ratio at higher IK14004 concentrations (FIG. 10). However, this was not associated with a statistically significant increase in the level of Foxp3 expression (FIG. 11).

This data demonstrates that RSKAKNPLYR-(2Adod)₄-NH₂ comprising increases the proportion of Treg cells.

### Example 7: RSKAKNPLYR-(2Adod)₄-NH₂ Reduces Tumour Area and Tumour Volume in Lung Cancer

The ability of a Lck activating polypeptide to modulate xenograft tumours in Lewis Lung Cancer (LLC) mice was examined.

Samples were in Bouin's solution for 24 hours and then placed into 70% ethanol prior to processing into FFPE blocks. Blocks were sectioned at 4 μm thickness, then mounted on Superfrost Plus slides before drying at 60° C. overnight. Sections were dewaxed in xylene and rehydrated through graded alcohols then stained using the following protocol:
1. Haematoxylin for 6 minutes;
2. Acid Alcohol brief wash;
3. Scotts Tap Water Substitute for 1 minute;
4. Aqueous Eosin Y for 20 seconds.

Slides were then dehydrated through graded alcohols and xylene, then scanned using Zeiss Axioscan Z1 slide scanner (whole slide).

There were 2 treatment groups with 8 mice in each: Vehicle (Water 100 μL, intraperitoneal injection, twice weekly; and IK14004 (400 μg in 100 μL Water, intraperitoneal injection, twice weekly.

Each data point shows the mean percentage of Lung area from 6 sections per mouse. Percentage area was determined by estimating the total area of each tumour from the regions drawn around to determine tumour number using Image J. Data shows a statistical difference between the two groups as determined by a two-tailed unpaired t-test **p<0.01.

The Lewis Lung Carcinoma cell line was cultured to approximately 70% confluency before cells were collected, counted and resuspended at 5×106/mL in sterile HBSS. Mice were injected subcutaneously with 5×105 cells (100 μl) into the right flank. Mice were randomly assigned to treatment groups 5 days after tumour cell implantation such that the average tumour size was approximately equal between the two groups. Test substance IK14004 (400 μg/200 mL), or, vehicle Sterile water 200 μL) were administered twice weekly (Monday and Thursday) via intraperitoneal (i.p) injections, from day 5 post tumour cell implantation, until the tumours reached an average of 10 mm in diameter in the vehicle treated group. Tumours were measured three times each week using digital callipers (Monday, Wednesday and Friday) by a scientist who was blinded to treatment groups. Once the mean tumour size for the vehicle group reached 10 mm in diameter, mice were sacrificed by cervical dislocation and tumour and spleens collected.

Tumour volume was estimated using the following calculation; 0.5×tumour length×width×width. Data are presented as individual data points with group mean+/−SEM, n=8. Groups were compared by two-way ANOVA with Sidak's multiple comparison, ****p<0.0001.

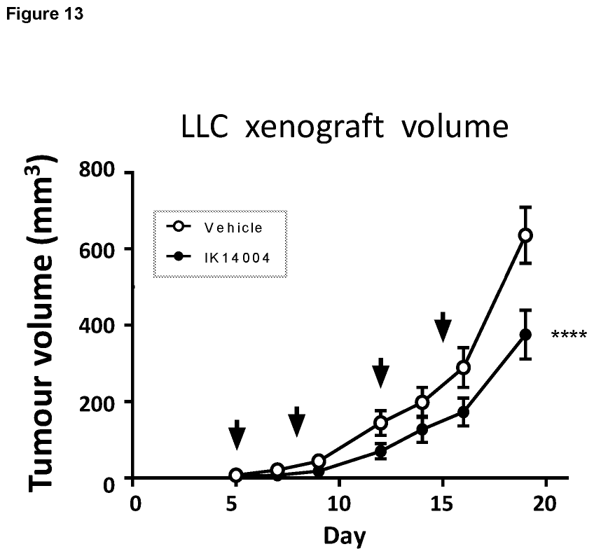

FIGS. 12 and 13 show RSKAKNPLYR-(2Adod)₄-NH₂ (“IK14004”) reduces xenograft tumour volume and tumour cell viability in the Lewis Lung Cancer (LLC) xenograft model.

This data indicates that in addition to maintaining homeostatic levels of IL-2 for the treatment of autoimmune disease, RSKAKNPLYR-(2Adod)₄-NH₂ reduces xenograft tumour volume and tumour cell viability.

To determine if RSKAKNPLYR-(2Adod)₄-NH₂ directly targets cancer cells, the effect of RSKAKNPLYR-(2Adod)₄-NH₂ on the of B16-F10 melanoma cells, and Lewis Lung Cancer cells was examined.

Cells (or Lewis Lung LL/2) were seeded into 96-well plates (1000 cells/well) in complete cell culture medium and allowed to attach for 24 h (37° C., 5% CO₂ in air). Next, an equal volume of either cell culture medium only, or 2×concentration of drug dissolved in cell culture medium, was added to each of 5 replicate wells (technical replicates) to expose cells to concentrations 0, 0.31, 0.63, 1.25, and 2.5 μM. In addition, doxorubicin at 2.5 μM was tested in 5 replicate wells as a positive control. Cells were cultured for a further 72 h in the presence of drug or control before the cell culture medium was removed and the attached cells fixed in ice-cold trichloroacetic acid. Fixed cells were stained with Sulforhodamine B (SRB), then washed with 1% acetic acid to remove unbound dye. The retained dye was solubilised in 10 mM Tris base solution, and the absorbance at 550 nm was measured, with the baseline (media only without cells) subtracted and the data normalised between the maximum proliferation (100%, cells with no drug) and the starting cell density (0%. cells before addition of drug). Each experiment was performed on two independent occasions (biological replicates) and data were analysed in GraphPad Prism 9 using non-linear fit of log (inhibitor) versus response.

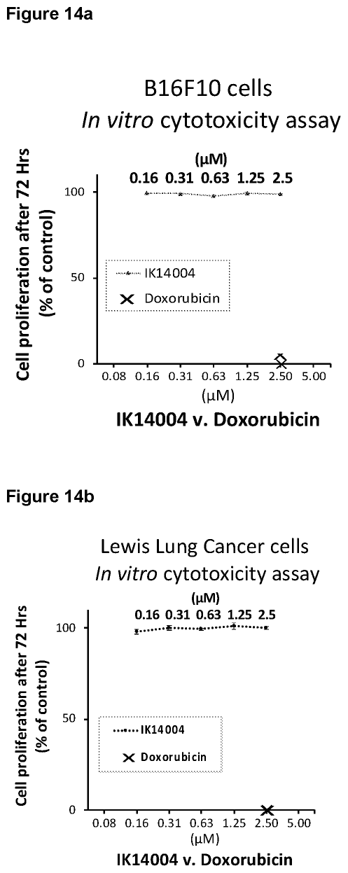

FIG. 14 demonstrates RSKAKNPLYR-(2Adod)₄-NH₂ did not case a decrease in cell proliferation, indicating that the effects of RSKAKNPLYR-(2Adod)₄-NH₂ demonstrated in FIGS. 12 and 13 are not caused by a direct effect of the peptide on tumour cells.

To determine if RSKAKNPLYR-(2Adod)₄-NH₂ acts via by immunomodulation, the effects of RSKAKNPLYR-(2Adod)₄-NH₂ on lung nodules, in a metastatic lung cancer model was examined.

In brief, peptide was supplied by as a pre-weighed samples in glass vials and was stored at −20° C. until use. The vial (9.8 mg) was resuspended in 2.45 mL of H2O to obtain a 4 mg/ml solution (for 400 μg dose level). This stock was diluted 1/10 in H2O for the 40 μg dose level. Solutions were then stored at 4° C. until use.

Twenty four female C57Bl/6 mice (WEHI, Age 8 weeks) were inoculated intravenously with 1×105 B16F10 cells in PBS on Aug. 5, 2017 (Day1).

Mice were randomised into three groups of 8 mice before being injected IP with 0.1 ml H20 or IK-14004 (40 μg or 400 μg/mouse). Treatments were given and mice were monitored for general health twice a week for 2 weeks. Mice were euthanized on Day 15, the lungs were removed, rinsed in PBS before being fixed in Fekete's solution. Lungs were then counted for the presence of lung tumour nodules. The data were analysed in GraphPad Prism using a one-way ANOVA followed by Dunnett's post-hoc test.

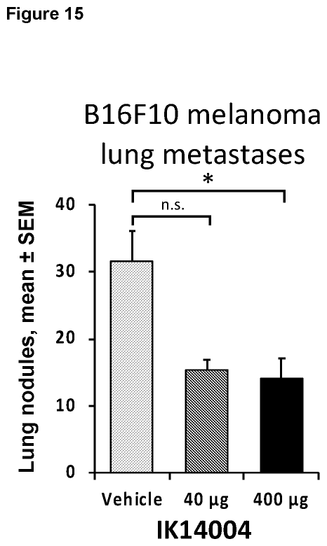

FIG. 15 demonstrates RSKAKNPLYR-(2Adod)₄-NH₂ (“IK14004”) reduces lung nodules, in a metastatic lung cancer model, as a result of immunomodulation.

These data indicate that IK14004 has no inhibitory effect on proliferation of either cancer cell lines at an in vitro concentrations of 2.5 uM which exceeds concentrations of IK14004 in either the lung or blood at 24 hours following administration of IK14004 to C57BL/6 mice based on pharmacokinetic data (not shown). Notably, the positive control, doxorubicin induced 100% kill in vitro against both cell lines. Taken together with the immunomodulating effects seen for 1K14004 at mid-nanomolar concentrations in vitro when tested on human immune cells it is postulated that IK14004-mediated tumour growth suppression in the murine models is due to its effects on the immune system and not direct effects on the cancer cells themselves.

### Example 8: RSKAKNPLYR-(2Adod)₄-NH₂ Enhances Expression of Receptors Required for Anti-Tumour Cytotoxicity by NK Cells

To examine the anticancer effects of IK14004 via immunomodulation, receptor expression on NK cells was examined.

FIGS. 16 and 17 demonstrate that RSKAKNPLYR-(2Adod)₄-NH₂ (“IK14004”) enhances IL-12 receptor expression on NK cells.

Accordingly, the anticancer effects of IK14004 (e.g. see FIGS. 12 to 15) are proposed to be mediated via its enhancement of receptor expression on NK cells required for anti-tumour cytotoxicity by NK cells, ie, enhanced IL-12 receptor expression that can respond to IL-12 produced by cancer cells which drives IFN-g production by NK cells

To examine the enhancement of receptor expression on NK cells required for anti-tumour cytotoxicity by NK cells, receptor expression on NK cells was further examined.

An important role for IL-2 is activation of the cytotoxic receptors on NK cells such as NKp44 and NKG2D that target cancer cells. For example, activation of NK cells by IL-2 induces expression of the natural cytotoxic receptor, NKp44, that is not expressed on resting NK cells and NKp44 is the first activating NK cell receptor to recognise a tumour growth factor.

FIG. 18 demonstrates RSKAKNPLYR-(2Adod)₄-NH₂ (“IK14004”) enhances NKp44 expression on NK cells. FIG. 19 demonstrates RSKAKNPLYR-(2Adod)₄-NH₂ (“IK14004”) enhances NKG2D receptor expression on NK cells.

These data indicate RSKAKNPLYR-(2Adod)₄-NH₂ enhances expression of receptors required for anti-tumour cytotoxicity by NK cells.

1. A method of treating or preventing an autoimmune disorder in a subject, said method comprising administering to the subject a therapeutically effective amount of a peptide comprising an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂.

2. A method according to claim 1, wherein the autoimmune disorder is a disorder associated with dysregulated IL-2 homeostasis.

3. A method according to claim 1 or claim 2, wherein the autoimmune disorder is an IL-2 mediated disorder

4. A method according to claim 1, wherein the autoimmune disease is associated with dysregulated IL-2 and/or IL-2Ralpha (CD25) production.

5. A method according to any one of claims 1 to 4, wherein the subject is deficient in IL-2 and IL-2Ralpha (CD25) production.

6. A method according to claim 1, wherein the autoimmune disorder is selected from the group consisting of allergic asthma, rheumatoid arthritis, multiple sclerosis, systemic lupus erythematosis and other lupus disorders, Type 1 insulin-dependent diabetes mellitus (IDDM), psoriasis, scleroderma, glomerular nephritis, ankylosing spondylitis, and GVHD.

7. A method according to any one of claims 1 to 6 wherein the method comprises selecting a subject for treatment by determining the levels of IL-2 and/or IL-2Ralpha (CD25) in the subject.

8. A method according to any one of claims 1 to 6 wherein the method comprises selecting a subject for treatment by determining the levels of IFNg and/or IL-12p40 in the subject.

9. A method according to any one of claims 1 to 8 wherein the subject has cancer.

10. A method according to claim 9 wherein the subject is receiving cancer therapy.

11. A method according to claim 10 wherein the subject is receiving checkpoint inhibitor therapy.

12. A method according to any one of 1 to 11 wherein the effective amount of a peptide comprising an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂ modulates the activity of Lck and/or G-protein signalling to maintain homeostatic levels of IL-2 in the subject.

13. A method according to claim 12 wherein the homeostatic levels of IL-2 are produced by cells selected from the group consisting of B cells, T cells and dendritic cells.

14. A method according to any one of claims 1 to 13 wherein the effective amount of a peptide comprising an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂ does not induce IFNg and/or IL-12p40.

15. A method according to any one of claims 1 to 14 wherein the therapeutically effective amount of a peptide comprising an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂ is administered orally and/or topically.

16. A method according to any one of claims 1 to 15 wherein the peptide consists of a peptide consists of an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂.

17. Use of therapeutically effective amount of a peptide comprising an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂ in a method of treating or preventing an autoimmune disorder in a subject.

18. Use of a peptide comprising an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂ in the manufacture of a medicament for treating an autoimmune disorder in a subject.

19. An oral dose form comprising an effective amount of a peptide comprising an amino acid sequence selected from the group consisting of RSKAKNPLYR-(2Adod)₂-NH₂, RSKAKNPLYR-(2Adod)₄-NH₂, rskaknplyr-(2Adod)₂-NH₂ and rskaknplyr-(2Adod)₄-NH₂ for the treatment of an autoimmune disorder in a subject

20. A method according to any one of claims 1 to 16 wherein the peptide is administered orally, or topically.

21. A method according to any one of claims 1 to 16 wherein the peptide is administered by injection.

22. A method according to any one of claims 1 to 16 wherein the peptide is administered by injection into a joint.

23. A method according to any one of claims 1 to 16 wherein the peptide is administered in the form of a pharmaceutical composition.

24. A method according to claim 23 wherein the pharmaceutical composition is administered to the subject simultaneously or sequentially with a cancer immunotherapy.
